(19) United States
(12) Patent Application Publication
(10) Pub. No.: US 2002/0106368 A1
(43) Pub. Date: Aug. 8, 2002

(54) Novel methods and compositions to upregulate, redirect or limit immune responses to peptides, proteins and other bioactive compounds and vectors expressing the same

(76) Inventors: Adrian Bot, San Diego, CA (US); Luis Dellamary, San Marcos, CA (US); Dan J. Smith, San Diego, CA (US); Catherine M. Woods, La Jolla, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/919,477

(22) Filed: Jul. 30, 2001

Related U.S. Application Data
(60) Provisional application No. 60/221,544, filed on Jul. 28, 2000.

(51) Int. Cl. A61K 39/395; A61K 38/22

(52) U.S. Cl. 424/130.1; 514/2

(57) ABSTRACT

Novel compositions are disclosed which can induce or enhance an immune response against foreign or self antigens (microbial or parasitic) or modulate (that can lead to suppression) the activity of pathogenic cells in inflammatory or autoimmune diseases. Compositions and methods are taught in how to limit the generation of an immune response against formulated peptides and proteins with application in antibody therapy or hormone replacement therapy. Methods of suppressing autoimmunity are also disclosed which use ligands for cellular receptors expressed on cells of the innate immune system and more specifically for down-regulation of autoimmune processes by either deletion or induction of anergy at the level of autoreactive T cells or by triggering active-suppressor T cells that down-regulate the activity of pathogenic cells.

### RELATED APPLICATIONS

The present application claims priority to U.S. Provisional Patent Application Serial No. 60/221,544, filed Jul. 28, 2000, the disclosure of which is incorporated herein by reference in its entirety.

### FIELD OF THE INVENTION

The present invention is generally related to various microparticle formulations and methods to control an immune response. More specifically, the present invention is directed to administration of formulated peptides or proteins formulated in microparticles which control (upregulate, redirect or limit) an immune response in a subject.

Novel compositions are disclosed which can induce or enhance an immune response against foreign antigens (microbial or parasitic) or modulate (that can lead to suppression) the activity of pathogenic cells in inflammatory or autoimmune diseases. Compositions and methods are disclosed in how to limit the generation of an immune response against formulated peptides and proteins with application in antibody or hormone replacement therapy. Methods of suppressing autoimmunity are also disclosed which use ligands for cellular receptors expressed on cells of the innate immune system and more specifically for down-regulation of autoimmune processes by either deletion or induction of anergy at the level of autoreactive T cells or by triggering active-suppressor T cells that down-regulate the activity of pathogenic cells.

### BACKGROUND OF THE INVENTION

Manipulation of an immune response is a constant goal of modern immunology. The process of vaccination represents induction or enhancement of immune responses against foreign antigens, associated with microbial or parasitic infections. In order to be effective, a vaccine should elicit certain arms of the immune response while not influencing other arms the immune response. For example, an effective vaccine against a viral infection should ideally expand the T1 controlled immunity, consisting of Th1 cells that produce IFN-γ, IL-2, LT-β, TNF-α; Tc1 cells that express perforin and granzymes and lyse virus-infected cells in a manner dependent on expression of MHC class I; and virus-neutralizing antibodies, represented by T1-controlled isotypes. In parallel, such a vaccine should not exacerbate Th2 immunity against foreign antigen, that may lead to potential negative interference with the effectors. As a whole, this process can be viewed as controlled induction of immune response (i.e. certain arms are induced, but other arms are spared). For example, live vaccines and killed or recombinant vaccines formulated with T1-promoting adjuvants have the ability to trigger Th1 responses but to a lesser extent Th2 responses. In contrast, killed vaccines formulated with alum which is a T2 driving adjuvant, have the ability to induce Th2 responses and to a lesser extent T1 immunity.

The profile of vaccines that are effective against virus infections is not necessarily compatible with suppression of T1-mediated inflammatory or autoimmune diseases. Diseases such as type 1 diabetes, multiple sclerosis, rheumatoid arthritis, autoimmune thyroiditis and psoriasis are mediated or associated with strong, harmful, localized or systemic immune responses against self antigens. Such immune responses are of T1 nature and are associated with unphysiological upregulation of cytokines such as IFN-γ, IL-2, LT-β and TNF-α. Effective vaccination or immunotherapy in context of such diseases should lead to selective reduction of pathogenic arms (i.e. autoreactive T1 cells), while non-pathogenic or regulatory arms should be expanded (i.e. Th2, Th3, Tr1 or other subsets). Thus, specific formulations of self antigens should be employed in order to achieve the goal of selectively inhibiting T1 responses and/or, enhancing non-pathogenic or regulatory responses. Such antigens may include antigen fragments or an antigen integrated into a recombinant molecule such as microbial antigens associated with microbes consisting of influenza, HIV, rotavirus, respiratory syncitial virus, hepatitis B, A, C, D, poliovirus, measles, mycobacteria tuberculosis, leishmania, listeria, pseudomonas, streptococcus and meningoccocus.

It becomes apparent that the goal of successful vaccination or immunotherapy is to rationally modulate immune responses, meaning to selectively upregulate or down-regulate arms of immunity that have protective or deleterious functions, respectively. Any methods to achieve the goal of controlling immunity would have clinical applications in the area of anti-microbial vaccination or immunotherapy of autoimmune diseases.

Type 1 diabetes, also known as insulin-dependent diabetes mellitus (IDDM), is an autoimmune disease resulting in the destruction of the insulin-producing islets of the pancreas. Data obtained from preclinical animal models of IDDM as well as clinical studies have implicated CD4⁺ and CD8⁺ autoreactive T cells as key effectors of islet cell destruction (J. F. Bach, Endocr. Rev., 1994). Despite the availability of insulin replacement therapy to maintain acceptable control of blood glucose levels, chronic insulin replacement therapy is still associated with major side effects including potential for acute hypoglycemia, chronic microvascular disease (retinopathy, nephropathy and neuropathy) and chronic macrovascular disease (heart disease and stroke) all resulting from the poor fine control of carbohydrate metabolism that can be attained with bolus injection of insulin (Simone et al., Diabetes Care, 22 Suppl. 2.: B7-B15, 1999). These side effects, combined with the high cost, the invasive nature of insulin therapy and the increasing prevalence of IDDM in the developed world, have led to efforts for finding alternative strategies including methods of preventing progression from the inciting autoreactive process to the irreversible loss of over 90% of the islet mass that correlates with clinical presentation of disease.

Studies of animal models for IDDM, particularly the nod mouse model, have shown that the pathogenic process starts with expansion of reactive T cells specific for dominant epitopes expressed by one or few islet-restricted antigens, but as the disease progresses, more determinants are involved via intra and intermolecular epitope spreading. Studies carried out in genetically manipulated animals demonstrated a critical role for the GAD-reactive T cells in the pathogenesis of disease (Baekkeskov et al., Autoimmunity, 15 Suppl. 24-26, 1993). However, cloning of autoreactive T cells from infiltrated islets and local lymph nodes strongly suggested an involvement of insulin-reactive T cells, particularly CD4⁺ as well as CD8⁺ T cells, that recognize dominant epitopes within insulin B 9-23 (Daniel et al., Eur. J Immunol., 25:1056-1062, 1995). Therefore, autoantigen-based immune therapy is an attractive strategy to suppress the autoimmune process since it may affect only the autoreactive T cells, thus leaving most of the T cell repertoire against non-self antigens intact to exert vital anti-microbial defense.

In contrast to anergy/deletion, the induction of active suppression may circumvent the multispecificity of the autoreactive process, by a mechanism that has been called “bystander suppression”. Observations generated during the early 1990's in nod mice injected with insulin antigen before the age of 4 weeks led to interest in this approach of delivering self antigens to modulate autoimmunity (reviewed by Simone et al., Diabetes Care, 22 Suppl. 2:B7-B15, 1999). These observations were paralleled by similar observations in humans at high risk of developing diabetes, prophylactically injected with hormonally active insulin (Keller et al., Lancet, 341:927-928, 1993). However, this early enthusiasm was tempered by two subsequent sets of observations: i) the difficulty of suppressing the autoimmune process by using antigen-based therapy administered during the later lymphocyte infiltration stages in development of the disease and, (ii) the potential of antigen-based modulation to actually aggravate the disease by expansion of pathogenic cells or even triggering expression of new effectors. Factors like antigen dose, route and schedule of administration, formulation, as well as more general factors such as the immune context of delivery and the precise stage of the autoimmune process, may all potentially impact the outcome in terms of disease suppression or aggravation. Thus, two critical issues must be solved before the field of antigen-based immune prophylaxis or therapy in autoimmune diseases like IDDM can be considered a viable strategy. First, there is a need to define regimens that can suppress the autoimmunity more effectively even when applied at later stages, not just during the initiating phase of insulitis. Second, new formulations have to be designed that reduce or eliminate the risk of aggravating the pathological process.

Membrane lectins that bind microbial or endogenous carbohydrate structure are expressed on antigen presenting cells like monocytes, macrophages and dendritic cells. A particular class of lectin-receptors are the mannose receptors, that mediate the internalization of mannosylated proteins and various pathogens like Mycobacteria (Schlesinger, L. S., J. Immunol., 150:2920-2930, 1993), Leishmania donovani (Wilson and Pearson, J. Immunol., 136:4681-4688, 1986), E. coli and pathogenic fungi like Candida albicans (Pacheco-Soares et al., Braz. J Med. Biol. Res., 25:1015-1024, 1992). The macrophage receptor responsible for the functions mentioned above was well defined and has a molecular weight of 150 kDa (Stahl, P. D., Curr. Opin. Immunol., 4:49-72, 1992). More recently, the expression of mannose-receptor isoforms was described on other cell types like dendritic cells (Reise Sousa et al., J. Exp. Med., 178:509-517, 1993; Avrameas et al., Eur. J. Immunol., 26:394-400, 1996).

Besides internalizing pathogens into the endo-lysosomal compartment of antigen presenting cells, mannose receptors may also play a role in triggering or modulating the production of soluble mediators such as cytokines. Cytokines like IL-1beta, IL-6, TNF-alpha and GM-CSF may be involved in the innate immune response against pathogenic fungi (Garner et al., J. Leukoc. Biol., 55:161-168, 1994; Yamamoto et al., Infect. Immun., 65:1077-1082, 1997). The mannose receptor has been shown to mediate the delivery of lipoglycan antigens to CD1b molecules, for the presentation to CD1-restricted CD8⁺ T cells, which are important in the defense against Mycobacteria (Prigozy et al., Immunity, 6:187-197, 1997). However, another study suggested that the TGF-beta production triggered by engagement of macrophage mannose receptors underlie the ability of virulent Mycobacteria strains to evade an immune response (Dahl et al., Infect. Immunol., 64:399-405, 1996).

In contrast to the data emerging that highlight the role of lectin receptors in antimicrobial defense or immune evasion by pathogens, little if anything is known regarding the involvement of lectin receptors and of mannose receptors in the regulation of autoimmune diseases in general, or the pathogenic processes that lead to autoimmune diabetes in particular. Further, it is believed that no studies approached the exploitation of mannose-receptor or lectin-receptor associated pathways in the prophylaxis or treatment of autoimmunity and more particularly, of autoimmune diabetes.

While animal models of IDDM have defined time courses for insulitis and early stage disease and onset of disease, this is not the case for the human population. Therefore, development of immunotherapeutic modulation of the autoreactive process driving islet destruction requires the development of reliable predictive markers for individuals at high risk for IDDM. The risk of disease in humans is related to the incidence of IDDM among relatives and expression of certain HLA alleles (i.e. DR3/4 is associated with disease progression whereas DQA1*0102 and DQB1*0602 are protective) (reviewed in Simone et al., Diabetes Care, 22 Suppl. 2:B7-B15, 1999). As described, the analysis of such factors of risk is well characterized and widely used. In addition, there is strong evidence that the titer and a broad spectrum of reactivity of circulating autoantibodies directed against islet-specific antigens (insulin, GAD, IA-2A and ICA-512) has increased predictive power (up to 75%) to identify individuals who will develop IDDM (Roll et al., Diabetes, 45:967-943, 1996; Kimpimaki et al., J. Clin. Endocrinol. Metab., 85:1126-1132, 2000). Taken together, these screening methods can identify individuals at high risk, before manifestation of the clinical signs of IDDM at which stage, the majority of islets have already been destroyed.

### SUMMARY OF THE INVENTION

There are three major aspects of the present invention:

1) The proposed formulations allow the control of a deleterious T cells response against self antigens, with application in prevention or suppression of an autoimmune, inflammatory disease. A related aspect is the control of response against organ-associated antigens, with applications in preventing/suppressing organ rejection and graft versus host reaction;

2) The proposed compositions and methods may be used to limit unwanted immune responses against peptides/proteins/compounds delivered by microparticles or formulations. This would have direct relevance for hormone replacement therapy; and,

3) The proposed compositions and methods may be used to selectively induce/enhance antibody responses against foreign (microbial) antigens or tumor associated antigens. This would have prophylactic application in area of vaccination or therapeutic implications (immunotherapy of cancers).

As stated previously, one aspect of the present invention refers to the prevention and control of a deleterious immune response against self T cell antigens, manifested by autoimmune or inflammatory diseases. Similar rationale can be applied to transplantation antigens that control the process of donor organ rejection. Previous methods to control ongoing autoimmune diseases and graft rejection relied on non-specific suppression of lymphocytes (corticosteroids or cyclosporin). Such methods were endowed with inherently low therapeutic window, due to toxic effects on the normal component of the immune repertoire. More recent approaches consist of ablation of cytokines or blockage of co-stimulatory receptors involved in inflammation or graft rejection. Such methods, although safer due to more restricted effects on normal immune repertoire, are still not specific and may be associated with limited efficacy due to redundant function of various immune mediators. The present invention proposes selective targeting of autoaggressive or pathogenic cells that are important to disease process. However, use of non-formulated peptides or non-engineered antigens to target autoaggressive cells has been hampered by limited efficiency of epitope presentation to T cells from this context. Various methods have been developed to circumvent this roadblock, consisting mainly of engineering recombinant constructs that bear disease-associated epitopes and receptor targeting motifs. Alternatively, therapeutic candidates that bind directly to T cell receptors have also been advanced.

The present invention proposes a novel method consisting in use of microparticle formulations for control of immune response against self antigens. Such formulations are biocompatible and flexible, allowing a wide range of manipulation of T cell responsiveness. The formulations described herein display novel immunological properties that may be of use in quenching ongoing deleterious T cell reactions. In addition, such formulations can be used to altogether prevent the onset of T cell reactions. The formulations of the present invention ideally contain one or more disease associated epitopes that facilitate the specific effect on immune system. Since the described formulations have the ability to induce regulatory cells that migrate in target organs, this novel technology may circumvent describing all or most of the disease-associated epitopes. For example, in a disease such as autoimmune (juvenile) diabetes, administration of dominant epitope such as insulin B chain formulated according to methods described below, may have the ability to suppress the pathogenic response of insulin B -specific T cells and to inhibit the T cell response against distinct pancreatic antigens (GAD 65 and HSP) via induction of regulatory cells. Definition of dominant epitopes associated with other autoimmune diseases (MBP, PLP and MOG in multiple sclerosis; HSP and collagen in rheumatoid arthritis) opens up the possibility of using such formulations in various instances. An important parameter of the present invention is flexibility, consisting in the ability to engineer various co-excipient profiles. Thus, such formulations can be improved in specific ways to increase the degree of control of immune response by associating biological response modifiers. Novel biological response modifiers (ligands for lectin receptors such as mannan) are described that can be used in context of microparticles or beyond the realm of microparticle formulations, to control deleterious T cell responses.

A second aspect of the present invention is the control of immune response to formulated active compounds such as peptides, proteins (immunoglobulin) and antigens in general. Previous methods of microparticle formulation did not take into account the immunological aspect to bioactive payload, in circumstances were non-immunological conditions were targeted. Only recently it was shown that even self peptides or proteins may trigger unwanted immune reactions, particularly when formulated in various types of structures. As specific examples, formulation and delivery of payloads peptide hormones such as insulin or of therapeutic immunoglobulins may lead to unwanted antibody responses against the payload. In case of insulin, such antibodies may interfere with the hormonal activity of hormone and if they are of IgE isotype, may mediate allergic reactions. In case of therapeutic immunoglobulins, such untoward responses may lead to limitation of activity as well as side effects such as immune complex—mediated diseases. The goal is to formulate such compounds in specific ways to minimize potentially deleterious immune reactions to the payload. The present application describes novel formulations that minimize such responses and thus can be applied in clinical field.

Another component of the present invention stemming from the ability to control immunity via specific formulations, consists of induction or amplification of antibody responses to foreign antigens. In particular cases such as prevention or treatment of microbial or parasitic infections, antibodies are of paramount importance. Using particular formulations that are described below, one can increase substantially the activation of B cells that recognize specific epitopes in context of antigens formulated in such microparticles. The immunoglobulin isotype or immunoglobulin-like molecule can be modulated by co-formulation of various biological response modifiers (an “immunoglobulin-like molecule” is defined as a construct containing domains, parts or epitopes of Ig origin. Antibodies triggered by formulated antigens bind to microbes and direct their internalization and degradation into phagocytic cells. Alternatively, antibodies may interfere with domains that bind to cellular receptors and facilitate infection. Lastly, antibodies may bind antigens expressed by infected cells and recruit effector cells that are bridged to such infected cells. This process (antibody dependent cellular cytotoxicity) results in selective elimination of infected cells. A similar rationale can be applied to induction of immune responses to tumor cells.

Another focus of the present invention is the surprising finding that certain ligands for lectin receptors such as the mannose receptor, when coformulated with self-antigens or administered independent of such antigens, have an unexpected effect in suppressing autoimmune diabetes. Specifically, methods are disclosed to suppress autoimmune processes using novel compositions based on phosphatides that include microbial or synthetic carbohydrates or analogues, as secondary excipients that bind lectin receptors expressed on the surface of cells of the innate immunity. Such immune modulating signals may lead to expression of IL-4, upregulation of IL-10, down-regulation of IL-12 and other modifications in cells of innate immunity, leading to overall suppression of the pathogenic reaction. Here, it is shown that mannans are a class of carbohydrate ligands that can modulate immune responses in a way that ultimately leads to suppression of IDDM. Methods are described that can be applied for in vitro (FACS analysis and cytokine production) and in vivo screening (suppression of disease in animal model of diabetes type 1) for ligands such as glycopeptides or glycolipids that bind to cellular receptors expressed on innate immune cells, leading to overall suppression of autoimmune disease.

The novel compositions disclosed comprise: a microparticle composed of surfactant (examples are shown where the surfactant is mainly a phosphatide), a carbohydrate that binds to lectin receptors on antigen presenting cells (mannan) and a self antigen, not hormonally active, such as insulin B chain.

Based on novel compositions. specific means are disclosed to enhance the efficiency and to ameliorate the safety profile of antigen-based immune prophylaxis or therapy of autoimmune diseases such as IDDM. The present invention also teaches how to circumvent the stage-dependency of the response to antigen-based immune therapy, by employing innate immune cells through engagement of non-antigen receptors. Compositions are disclosed that suppress autoimmunity when applied both during the early initiation phase of insulitis, as well as during later pathogenic stages associated with active islet destruction. Using these formulations for antigen-based immune modulation, it is shown that such methods do not necessarily lead to the depletion of autoreactive cells but may actually result in expansion of autoreactive regulatory cells with a suppressor effect on disease. More generally, the use of such compositions is proposed for the purpose of limiting as well as modulating immune responses to various therapeutic compounds. Methods for including excipients that can limit untoward immune reactions are of key importance when considering particulate drug delivery platforms for systemic delivery of therapeutic proteins and peptides via the respiratory mucosa.

Preferred compositions of the present invention associated with limited immunity are obtained by spray drying at temperatures of less than 100° C. (preferably less than 60° C.). It is also contemplated that the particles (microparticles) of the present invention can be obtained by homogenization (any type of colloidal suspension-emulsion, double emulsion, bicontinuous emulsions, micellar, isotropic solutions, etc), lyophilization, precipitation, solvent evaporation, flash evaporation. However, the preferred method of manufacturing the microparticles of the present invention is by spray-drying.

The compositions may be 1-80% surfactant by weight (e.g. surfactants may be selected from phosphatides, non-ionic surfactants, cationic surfactants, anionic surfactants and proteins, amino acids and oligoaminoacids that are known to exhibit surfactant properties); 10-50% excipient by weight (excipients may be selected from carbohydrates, salts, proteins and/or synthetic polymers); 0-80% bioactive substance by weight and optionally a molar ratio of metal ion to phosphatides of 0-2. Other ranges of surfactants may 0.1-80%, 20-80%, 40-80%, 60-80%, 0.1-20%, and 0.1-10% by weight. Other ranges of excipients may be 0.1-10%, 20-50%, 30-50%,40-50% and 50-60%.

Preferred surfactants include phosphatides: homo and heterochain phosphatidylcholines (PC's), phosphatidylserines (PS's), phosphatidylethanolamines (PE's), phosphatidylglycerols (PG's), phosphatidylinositols (PI's), sphingomyelins, gangliosides, 3-Trimethylammonium-Propane phosphatides (TAP's) and Dimethylammonium-Propane phosphatides (DAP's), having hydrocarbon chain length ranging from 5 to 22 carbon atoms. One (lysophosphatides) or double chain surfactants are also contemplated. The phosphatides may be hydrogenated, unsaturated or partially hydrogenated. The most preferred phosphatides are natural phosphatides and hydrogenated phosphatides derived from soy or egg, partially hydrogenated phosphatides derived from soy and egg, DiC18PC, DiC16PC, DiC14PC, DiC8PC DiC6PC, DiC16PS DiC14PS, DiC8PS and DiC6PS.

Contemplated non-ionic surfactants include poloxamers, tweens, tritons, PEG's, sugar esters. Most preferable non-ionic surfactants are poloxamer 188, poloxamer 407, tween 80, PEG 1540 cetyl alcohol and tyloxapol. Cationic surfactants may include benzalkonium chloride. Anionic surfactants may be selected from the cholate and deoxycholate family, like CHAPS (MERCK index 11 ed., monography pg. 2034), taurocholate, deoxytaurocholate, or phosphate fatty acid salts such as dicetyl phosphate. Other surface active compounds include albumin, leucine, oligopeptides, oligoleucine, oligoalanine and saponins (for a further listing see Gower's handbook of industrial surfactants 1993, pages 885-904, ISBN 0566074575 which is hereby incorporated by reference).

Preferred excipients for the compositions include carbohydrates such as lactose, mannitol, mannose, sorbitol, sucrose, trehalose, saponins etc. Proteins include albumin (human, egg or bovine), oligopeptides, oligoleucine, oligoalanine, etc. Synthetic polymers include PEGs and Poloxamers, etc. Other excipients include osmotic agents such as NaCl, KCl, magnesium chloride, calcium chloride, zinc chloride, etc. and buffer systems like PBS, acetate, citrate, tris, etc.

Preferred metal ions include metal ions or salts from groups IIa, IIIa and metal ions from atomic numbers 21-30; 39-48, 57-80 and 89-106. The preferred metal ions are calcium, magnesium, aluminum and zinc.

Some compositions of the present invention include the following compositions:

1) A microparticle composition for controlling an immune response by downregulating a pathothegic arm of the immune system, or upregulating the suppressor arm of the immune system, or simultaneously downregulating the pathogenic arm and upregulating the suppressor arm of the immune system comprising:

a surfactant or mixture of surfactants comprising approximately 1-80% of the weight of the total microparticle composition wherein the surfactant can be selected from hydrogenated, unsaturated or partially hydrogenated phosphatides which can optionally be derived from soy or egg, examples of phosphatides include homo- and heterochain PC's, PS's, PE's, PG's, PI's, sphingomyelins, gangliosides, TAP's, DAP's, DiC18PC, DiC16PC, DiC14PC, DiC8PC DiC6PC, DiC16PS DiC14PS, DiC8PS, DiC6PS, non-ionic surfactants (e.g., poloxamers, tweens, tritons, PEGs, sugar esters, poloxamer 188, poloxamer 407, tween 80, PEG 1540, cetyl alcohol, tyloxapol, benzalkonium chloride, cholate, deoxycholates, CHAPs, taurocholate, deoxytaurocholate, phosphate fatty acid salts like dicetyl phosphate), cationic surfactants, proteins, amino acids and oligoaminoacids;

at least one excipient which may include carbohydrates (e.g., hetastarch, starches, lactose, mannitol, mannose, inulin, mannan, sorbitol, galactitol, sucrose, trehalose, raffinose, maltose, glucose, cellulose and derivatives, pectins, dextrans, dextrins, chitosan, chitin, mucopolysaccharides, chondroitin sulfate and saponins), polyols, salts, proteins (human, egg or bovine albumin, chollagen, oligopeptides, oligoleucine, oligoalanine, gelatin, and glycoproteins) and synthetic polymers (e.g., PLGA's, polylactides, polyglycolides, PVA's, PVP's, polyacrylics, carbomers, polyanhydrides, polyphosphoethers, polyurethanes, polyesters and polyphosphazenes)

and,

at least one foreign or self-antigen wherein the antigen may be a peptide or protein hormone (e.g., insulin) or an immunoglobulin or an immunoglobulin-like molecule, other possible antigens may be a formulated antigen, antigen fragments or antigen(s) integrated into a recombinant molecule and may be disease associated and may be insulin, GAD, HSP, collagen, MBP, PLP, and MOG or may be microbial associated and may be influenza, HIV, rotavirus, respiratory syncitial virus, hepatitis B, A, C, D, poliovirus, measles, mycobacteria tuberculosis, leishmania, listeria, pseudomonas, streptococcus and meningoccocus.

2) A microparticle composition for the treatment of an autoimmune disorder (e.g., type 1 diabetes) comprising:

at least one surfactant (e.g., phosphatide, phosphatidylcholine, partially or hydrogenated phosphatidylcholine from egg or soy) wherein the at least one surfactant comprises approximately 1-80% of the total weight of the microparticle composition;

a carbohydrate (e.g., mannan) that binds to the lectin receptors (e.g., mannose receptor) on antigen presenting cells;

and

an antigen (e.g., insulin).

3) A microparticle composition for the treatment of an autoimmune disorder comprising:

a surfactant (e.g., phosphatide) or surfactant mixture comprising approximately 1-80% of the total weight of the microparticle composition wherein the surfactant may be partially or hydrogenated phosphatidylcholine from egg or soy;

a carbohydrate (e.g., mannan) that binds to the lectin receptor (e.g., mannose receptor) on antigen presenting cells comprising approximately 1-60% of the total weight of the microparticle composition.

4) A microparticle composition for delivering a bioactive substance where it is desired to limit the immune response to the bioactive substance comprising:

at least one water soluble surfactant which may be a phosphatide, non-ionic surfactant, anionic surfactant, cationic surfactant, protein, amino acid and oligoaminoacid;

at least one water soluble excipient comprising a weight ratio of 1-90% of the total weight of the composition wherein the water soluble excipients which may be lactose, mannitol, mannose, sorbitol, galactitol, sucrose, trehalose, raffinose, maltose, glucose, saponins, osmotic agents such as sodium chloride, potassium chloride, calcium chloride, magnesium chloride, zinc chloride, buffers such as PBS, acetate, citrate, TRIS and amino acids such as glycine and alanine;

a bioactive substance (e.g., insulin);

and may further have a metal ion.

5) A microparticle composition for delivering a bioactive substance where it is desired to enhance the immune response to the bioactive substance comprising:

a surfactant or mixture of surfactants which may be selected from phosphatides, non-ionic surfactants (e.g., tyloxapol), anionic surfactants, cationic surfactants, proteins, amino acids and oligaminoacids;

an excipient which may be selected from starches, lactose, mannitol, mannose, inulin, mannan, sorbitol, galactitol, sucrose, trehalose, raffinose, maltose, glucose, cellulose and derivatives, pectins, dextrans, dextrins, chitosan, chitin, mucopolysaccharides, chondroitin sulfate, saponins osmotic agents such as sodium chloride, potassium chloride, calcium chloride, magnesium chloride, zinc chloride, buffers such as PBS, acetate, citrate, TRIS, amino acids such as glycine and alanine, human, egg or bovine albumin, chollagen, oligopeptides, oligoleucine, oligoalanine, gelatin, glycoproteins, PLGA's, polylactides, polyglycolides, PVA's, PVP's, polyacrylics, carbomers, polyanhydrides, polyphosphoethers, polyurethanes, polyesters and polyphosphazenes;

and a bioactive substance (e.g., insulin, nucleic acids, nucleotides, peptides and proteins) for inducing an immune response; and,

the microparticle may optionally contain a metal ion.

Any of the compositions taught herein may be administered to the respiratory tract by liquid dose instillation, nebulization, aerosolization, dry powder inhalation and metered dose instillation. Any of the of the compositions taught herein may also be administered intravenously, subcutaneously, intramuscularly, intradermally, transdermally, and intraperitoneally.

Other aspects of the present invention are set forth in the following numbered paragraphs:

1. A microparticle composition for controlling an immune response by downregulating a pathothegic arm of the immune system, or upregulating the suppressor arm of the immune system, or simultaneously downregulating the pathogenic arm and upregulating the suppressor arm of the immune system comprising:

a surfactant or mixture of surfactants comprising approximately 1-80% of the weight of the total microparticle composition;

at least one excipient selected from the group consisting of carbohydrates, polyols, salts, proteins and synthetic polymers; and,

at least one antigen.

2. The microparticle composition of Paragraph 1 wherein the antigen is selected from the group consisting of foreign antigens and self-antigens.

3. The microparticle composition of Paragraph 1 wherein the antigen is a protein antigen.

4. The microparticle of Paragraph 3 wherein the protein antigen is an immunoglobulin or an immunoglobulin-like molecule.

5. The microparticle composition of Paragraph 1 wherein the microparticle composition suppresses an ongoing deleterious immune response.

6. The microparticle composition of Paragraph 1 wherein the microparticle composition prevents a deleterious immune response.

7. The microparticle composition of Paragraph 1 wherein the microparticle composition prevents, suppresses or limits an immune response against a delivered bioactive payload.

8. The microparticle composition of Paragraph 7 wherein the payload is a peptide hormone.

9. The microparticle composition of Paragraph 1 wherein the microparticle composition enhances induction of a Th2 cellular response.

10. The microparticle composition of Paragraph 9 wherein the microparticle composition induces an enhanced expression of IL-4.

11. The microparticle composition of Paragraph 1 wherein the microparticle composition enhances induction of a humoral response.

12. The microparticle composition of Paragraph 11 wherein the humoral response is directed against a foreign epitope or tumor associated antigens.

13. The microparticle of Paragraph 12 wherein the foreign epitope is selected from the group consisting of microbial epitopes and parasitic epitopes.

14. The microparticle composition of Paragraph 1 wherein the microparticle is compatible with deep lung delivery.

15. The microparticle of Paragraph 1 wherein the surfactant is selected from the group consisting of phosphatides, non-ionic surfactants, cationic surfactants, proteins, amino acids and oligoaminoacids.

16. The microparticle of Paragraph 8 wherein the phosphatide surfactant is chosen from the group consisting of homo and heterochain PC's, PS's, PE's, PG's, PI's, sphingomyelins, gangliosides, TAP's and DAP's, having one or two hydrocarbon chain length ranging from 5 to 22 carbon atoms.

17. The microparticle of Paragraph 8 wherein the phosphatides may be hydrogenated, unsaturated or partially hydrogenated.

18. The microparticle of Paragraph 17 wherein the phosphatides are phosphatides derived from soy or egg.

19. The microparticle composition of Paragraph 15 wherein the phosphatide is selected from the group consisting of DiC18PC, DiC16PC, DiC14PC, DiC8PC DiC6PC, DiC16PS DiC14PS, DiC8PS and DiC6PS.

20. The microparticle composition of Paragraph 15 wherein the non-ionic surfactant is selected from the group consisting of poloxamers, tweens, tritons, PEGs, and sugar esters.

21. The microparticle composition of Paragraph 15 wherein the non-ionic surfactant is selected from the group consisting of poloxamer 188, poloxamer 407, tween 80, PEG 1540, cetyl alcohol and tyloxapol.

22. The microparticle composition of Paragraph 15 wherein the non-ionic surfactant is selected from the group consisting of benzalkonium chloride, cholate, deoxycholates, CHAPs, taurocholate, deoxytaurocholate, phosphate fatty acid salts like dicetyl phosphate.

23. The microparticle composition of Paragraph 1 wherein the at least one surfactant is selected from the group consisting of albumin, leucine, oligopeptides, oligoleucine, oligoalanine and saponins.

24. The microparticle composition of Paragraph 1 wherein the carbohydrate excipient is selected from the group consisting of include hetastarch, starches, lactose, mannitol, mannose, inulin, mannan, sorbitol, galactitol, sucrose, trehalose, raffinose, maltose, glucose, cellulose and derivatives, pectins, dextrans, dextrins, chitosan, chitin, mucopolysaccharides, chondroitin sulfate and saponins.

25. The microparticle composition of Paragraph 1 wherein the protein excipient is selected from the group consisting of human, egg or bovine albumin, chollagen, oligopeptides, oligoleucine, oligoalanine, gelatin, and glycoproteins.

26. The microparticle composition of Paragraph 1 wherein the synthetic polymer excipient is selected from the group consisting of PLGA's, polylactides, polyglycolides, PVA's, PVP's, polyacrylics, carbomers, polyanhydrides, polyphosphoethers, polyurethanes, polyesters and polyphosphazenes.

27. The microparticle composition of Paragraph 1 wherein the microparticle composition is delivered to the respiratory tract.

28. The microparticle composition of Paragraph 1 wherein the antigen is insulin.

29. The microparticle composition of Paragraph 1 wherein the formulated antigen, antigen fragment or antigen integrated into a recombinant molecule is disease associated and selected from the group consisting of insulin, GAD, HSP, collagen, MBP, PLP, and MOG.

30. The microparticle composition of Paragraph 1 wherein the formulated antigen, antigen fragment or antigen integrated into a recombinant molecule is microbial associated selected from the group of microbes consisting of influenza, HIV, rotavirus, respiratory syncitial virus, hepatitis B, A, C, D, poliovirus, measles, mycobacteria tuberculosis, leishmania, listeria, pseudomonas, streptococcus and meningoccocus.

31. The microparticle composition of Paragraph 1 further comprising tyloxapol.

32. A microparticle composition for the treatment of an autoimmune disorder comprising:

at least one surfactant wherein the at least one surfactant comprises approximately 1-80% of the total weight of the microparticle composition;

a carbohydrate that binds to the lectin receptors on antigen presenting cells; and

an antigen.

33. The microparticle composition of Paragraph 32 wherein the autoimmune disorder is type 1 diabetes.

34. The microparticle composition of Paragraph 32 wherein the main surfactant is a phosphatide.

35. The microparticle of Paragraph 32 wherein the main surfactant is a phosphatidylcholine.

36. The microparticle composition of Paragraph 35 wherein the main surfactant is a partially or hydrogenated phosphatidylcholine from egg or soy.

37. The microparticle composition of Paragraph 32 wherein the lectin receptor is a mannose receptor.

38. The microparticle composition of Paragraph 37 wherein the carbohydrate is mannan.

39. The microparticle composition of Paragraph 32 wherein the antigen is insulin.

40. The microparticle composition of Paragraph 39 wherein the antigen is insulin B chain.

41. The microparticle composition of Paragraph 32 wherein the carbohydrate includes microbial or synthetic carbohydrates or derivatives.

42. A method of treating a patient suffering from Type 1 diabetes by administration of a therapeutically effect amount of microparticles as described in Paragraph 32.

43. A method if treating a patient suffering from Type 1 diabetes by administration of a therapeutically effective amount of microparticles as described in Paragraph 38.

44. The method of Paragraph 42 wherein the patient is treated during the early initiation phase of insulitis.

45. The method of Paragraph 42 wherein the patient is treated during later pathogenic stages of Type 1 diabetes associated with active islet cell destruction.

46. A method of enhancing the Th2 response of an individual suffering from an autoimmune disorder comprising administration of a therapeutically effective amount of the microparticle composition of Paragraph 32.

47. A method of enhancing the Th2 response of an individual suffering from an autoimmune disorder comprising administration of a therapeutically effective amount of the microparticle composition of Paragraph 40.

48. A method of enhancing the IL-4 production of an individual suffering from an autoimmune disorder comprising administration of a therapeutically effective amount of the microparticle composition of Paragraph 32.

49. A method of tolerizing pathogenic T-cells in an individual suffering from autoimmune diabetes comprising administration of a therapeutically effective amount of the microparticle composition of Paragraph 37.

50. A method of preventing the onset of Type 1 diabetes by administration of a therapeutically effective amount of the microparticle composition of Paragraph 32.

51. A microparticle composition for the treatment of an autoimmune disorder comprising:

a surfactant or surfactant mixture comprising approximately 1-80% of the total weight of the microparticle composition; and

a carbohydrate that binds to the lectin receptors on antigen presenting cells comprising approximately 1-60% of the total weight of the microparticle composition.

52. The microparticle composition of Paragraph 51 wherein the main surfactant is a phosphatide.

53. The microparticle composition of Paragraph 51 wherein the lectin receptor is a mannose receptor.

54. The microparticle composition of Paragraph 51 wherein the carbohydrate is mannan.

55. The microparticle composition of Paragraph 51 wherein the main surfactant is a partially or hydrogenated phosphatidylcholine from egg or soy.

56. A method of preventing the development of type 1 diabetes comprising administering a therapeutically effective amount of the microparticle compositions of Paragraph 51.

57. A method of preventing the development of type 1 diabetes comprising administering a therapeutically effective amount of the microparticle composition of Paragraph 53.

58. A method of preventing the development of type 1 diabetes comprising administering a therapeutically effective amount of the microparticle composition of Paragraph 54.

59. A method of enhancing the Th2 response of an individual suffering from an autoimmune disorder comprising administration of a therapeutically effective amount of the microparticle composition of Paragraph 51.

60. A method of tolerizing pathogenic T-cells in an individual suffering from autoimmune diabetes comprising administration of a therapeutically effective amount of the microparticle composition of Paragraph 51.

61. A microparticle composition for delivering a bioactive substance where it is desired to limit the immune response to the bioactive substance comprising:

a water soluble surfactant selected from the group consisting of: phosphatides, non-ionic surfactants, anionic surfactants, cationic surfactants, proteins, amino acids and oligoaminoacids;

a water soluble excipient comprising a weight ratio of 1-90% of the total weight of the composition wherein the water soluble excipients is selected from the group consisting of lactose, mannitol, mannose, sorbitol, galactitol, sucrose, trehalose, raffinose, maltose, glucose, saponins, osmotic agents such as sodium chloride, potassium chloride, calcium chloride, magnesium chloride, zinc chloride, buffers such as PBS, acetate, citrate, TRIS and amino acids such as glycine and alanine; and,

a bioactive substance.

62. The microparticle composition of Paragraph 61 wherein the bioactive substance is insulin.

63. The microparticle composition of Paragraph 61 wherein the microparticle composition is administered to the respiratory tract.

64. The microparticle composition of Paragraph 61 wherein the microparticle composition results in a less retentive microparticle which has a high dissolution rate in water.

65. The microparticle composition of Paragraph 61 wherein the microparticle composition results in a high dissolution rate and a low clearance by phagocytes in the respiratory tract.

66. The microparticle composition of Paragraph 61 wherein the microparticle composition limits the Th2 immune response.

67. The microparticle of Paragraph 61 further comprising a metal ion.

68. The microparticle composition of Paragraph 61 further comprising tyloxapol.

69. The microparticle of Paragraph 61 wherein the surfactant or mixture of surfactants is present in an amount of approximately 1-80% of the total weight of the microparticle composition.

70. A microparticle composition for delivering a bioactive substance where it is desired to enhance the immune response to the bioactive substance comprising:

a surfactant or mixture of surfactants selected from the group consisting of phosphatides, non-ionic surfactants, anionic surfactants, cationic surfactants, proteins, amino acids and oligaminoacids;

an excipient selected from the group consisting of starches, lactose, mannitol, mannose, inulin, mannan, sorbitol, galactitol, sucrose, trehalose, raffinose, maltose, glucose, cellulose and derivatives, pectins, dextrans, dextrins, chitosan, chitin, mucopolysaccharides, chondroitin sulfate, saponins osmotic agents such as sodium chloride, potassium chloride, calcium chloride, magnesium chloride, zinc chloride, buffers such as PBS, acetate, citrate, TRIS, amino acids such as glycine and alanine, human, egg or bovine albumin, chollagen, oligopeptides, oligoleucine, oligoalanine, gelatin, glycoproteins, PLGA's, polylactides, polyglycolides, PVA's, PVP's, polyacrylics, carbomers, polyanhydrides, polyphosphoethers, polyurethanes, polyesters and polyphosphazenes;

and a bioactive substance for inducing an immune response.

71. The microparticle composition of Paragraph 70 wherein the microparticle composition is administered to the respiratory tract.

72. The microparticle composition of Paragraph 70 wherein the bioactive substance is insulin.

73. The microparticle composition of Paragraph 70 wherein the microparticle composition results in a retentive microparticle which slows the release of the bioactive substance.

74. The microparticle composition of Paragraph 70 wherein the microparticle composition results in aggregation and slows clearance by phagocytes in the respiratory tract.

75. The microparticle composition of Paragraph 70 wherein the microparticle composition increases the Th2 immune response.

76. The microparticle of Paragraph 70 further comprising a metal ion.

77. The microparticle composition of Paragraph 70 wherein a non-ionic surfactant is added to increase the release rate of the bioactive substance.

78. The microparticle composition of Paragraph 77 wherein the non-ionic surfactant is tyloxapol.

79. The microparticle composition of Paragraph 70 wherein the bioactive substance is selected from the group consisting of nucleic acids, nucleotides, peptides and proteins.

80. The microparticle composition of Paragraph 1 wherein the microparticle composition can be administered to the respiratory tract by liquid dose instillation, nebulization, aerosolization, dry powder inhalation and metered dose instillation.

### BRIEF DESCRIPTION OF THE DRAWINGS

FIGS. 1A-1B illustrates a surface electron microscopy of SDLM;

FIG. 2 shows an Andersen cascade impactor analysis of the SDLM of the present invention;

FIG. 3 shows enhancement of a Th2 response by antigen formulated in SDLM;

FIG. 4 demonstrates that a slow release effect is responsible for enhancement of a Th2 response;

FIGS. 5A and 5B show increase of Th2-dependent IgG1 response against formulated antigen;

FIGS. 6A-6B show dependency of antibody response to formulated antigen on CD3⁺T cells;

FIGS. 7A-7D illustrate dependency of Th2 and antibody response to formulated antigen and on expression of MHC class II molecules;

FIG. 8 illustrates enhanced B cell responses to formulated antigen in the absence of functional T cells;

FIG. 9 shows enhanced ability of APC purified from mice injected with formulated antigen to stimulate memory T cells;

FIGS. 10A-10B show that formulation into SDLM is a method to aggregate immunoglobulins and other proteins;

FIGS. 11A-11C show that repeated administration of formulated IgG into mice results in expansion of IL-4 producing T cell population;

FIG. 12 shows enhanced Th2-dependent IgG1 responses subsequent to administration of autogenic immunoglobulin formulated in SDLM;

FIGS. 13A and 13B show the immunogenicity of an immunoglobulin recombinant construct bearing a T cell epitope and formulated into SDLM;

FIGS. 14A-14C show modification of splenic autoreactive T cell response in non-obese diabetic mice (NOD) by administration of self-antigen formulated into spray dried microparticles;

FIGS. 15A-15C show a modified lymph node profile of T cells in non-obese diabetic animals treated with spray dried particles loaded with insulin B chain;

FIGS. 16A-16B show the suppression of autoimmune diabetes by administration of self-antigen formulated into SDLM;

FIG. 17 shows the kinetics of disease in IL-4 deficient non-obese diabetic mice treated with insulin B formulated into various SDLM;

FIGS. 18A-18B show substantiation of Th2 response to antigen coformulated with mannan into SDLM;

FIGS. 19A-19C show the induction of IL-4 producing T cells by carbohydrate conjugates of bovine serum albumin (“BSA”);

FIGS. 20A-20B illustrate the suppression of autoimmune diabetes by administration of self-antigen coformulated with mannan in spray dried particles (SDLM);

FIGS. 21A-21B show modification of autoreactive T cell immunity by administration of insulin B coformulated with mannan into SDLM to non-obese diabetic mice;

FIGS. 22A-22B illustrate the protective effect of spray dried particles loaded with mannan and devoid of self antigen on disease in non-obese mice;

FIGS. 23A-23B illustrate the effect of mannan-SDLM on the profile of autoreactive T cells from NOD mice;

FIG. 24 shows data from internalization of fluorescent particles (“ret”=retentive and “non-ret”=non-retentive) by bronchoalveolar phagocytes;

FIG. 25 shows limited antibody response to non-retentive (“Non-ret”) particles compared to retentive (“Ret”) formulation;

FIG. 26 shows limited antibody response to non-retentive (“Non-ret”) particles compared to retentive (“Ret”) formulation;

FIG. 27 shows release of IgG from spray dried formulations;

FIG. 28 shows the release rate of IgG from powdered compositions;

FIG. 29 shows the measurement of the “particle” characteristics of bovine IgG compositions;

FIG. 30 shows the release rates of spray dried compositions containing bovine IgG as an example of a bioactive compound; and

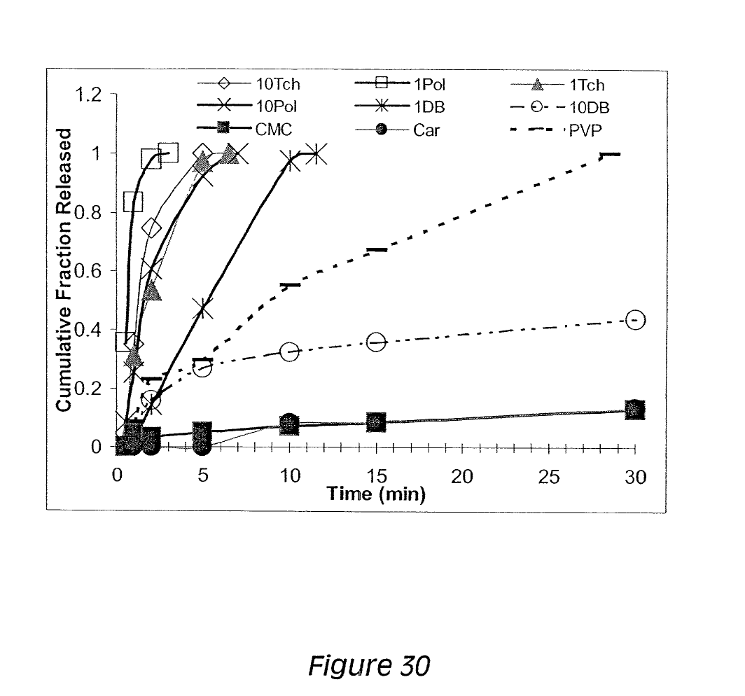

FIG. 31 shows the release rates of lyophilized compositions containing insulin and the effect of surfactant concentration on release rate.

### DETAILED DESCRIPTION OF THE INVENTION

Disclosed herein are microparticle-bioactive agent formulations for delivery of a bioactive agent to the respiratory tract or other parts of the body, that based upon need, allow the induction or enhancement of a desired immune response or to avoid or limit an immune response against the delivered bioactive agent.

A microparticle formulation containing a bioactive compound (e.g., peptides, proteins, nucleotides) delivered to the respiratory tract is capable of inducing an immune response if the bioactive agent is still attached or incorporated to the matrix of the microparticle. If the bioactive substance is not effectively released from the microparticle matrix (that is, the composition is expressing “particle-like” characteristics) within one hour after delivery, an immune response can be induced against the delivered antigen. In order to avoid particle-like characteristics and avoid or lessen an immunological response against the delivered bioactive agent, surface active components such as hydrosoluble small to medium molecular weight excipients can be used to formulate the bioactive compound and create what can be called a non-retentive particle.

The role of a surfactant is to: 1) increase the spreadability of the formulation; 2) act as a wetting agent; 3) reduce interactions between particles of a colloidal nature; and 4) reduce the water-air interface stress of some bioactive compounds that will cause loss of bioactivity. With non-retentive particles, the surfactant should have reduced amounts of multilamellar or multiple stacked amphiphiles that could resemble antigens (the opposite is true if retentive particles are intended). The surfactant or surfactant mixture for non-retentive particles should have a high HLB (“hydrophilic-lipophilic balance”) such as water soluble surfactants. A mixture of surfactants could also be a combination of a low HLB surfactant with a high HLB surfactant which usually acts overall as a high number HLB surfactant. For example, DPPC formulated with a non-ionic surfactant, such as tyloxapol, will give rise to a surfactant mixture that has reduced particle-like characteristics. Preferred surfactants of the present invention include non-toxic surfactants at a concentration needed to achieve the purpose of lessening particle-like characteristics and avoiding and/or controlling the immune response. Also, the properties of surfactants having low water solubility and low diffusion rate can be modified by the addition of small amounts of high water soluble and high diffusion surfactants to obtain the desired effect.

Specific excipients may also be used to avoid or to lessen an immune response. As with surfactants, the preferred stabilizing excipients for formulations for avoiding or lessening an immune response are small to medium molecular weight compounds with relatively high water solubility and high diffusion rates. Less favorable excipients are those excipients which have a tendency to swell in water and promote formation of particle-like structures (the opposite is true if retentive particles are intended).

Because particle size is a limiting factor and therefore often the focus for delivery of bioactive compounds to the respiratory tract, little attention has been given to the nature of the particle when peptides, proteins, nucleotides and other bioactive compounds are delivered to or via the respiratory tract. The trend has been to produce controlled release formulations for the systemic or local delivery of proteins and nucleotides, that if delivered to the respiratory tract, can induce an immune response against the bioactive compound, which may not be desirable. It is shown in the present application that the nature of the particle (“particle” is defined herein as being of colloidal origin, that is, solid in gas; liquid in liquid; liquid in gas; solid in liquid; etc.) is of crucial importance if it is desired to limit or enhance an immune response.

### EXAMPLE 1

### Spray Dried Formulations of Antigens and Antigenic Immunoglobulins

Preparation A was comprised of a liposome suspension of 0.37 g of dipalmitoylphosphatidylcholine (DPPC) dispersed in 23 g of hot DI water with a T-25 Ultraturrax at 9000 rpm for about 5 min. The coarse liposomes were homogenized under high pressure (18,000 psi) for 5 discrete passes with an Avestin Emulsiflex C5. Preparation B contained 0.1 g of CaCl₂.2H₂O, 0.012 g of tyloxapol and 0.36 g of lactose monohydrate. Preparation A was added to dissolve all of the ingredients in preparation B, now called preparation (A+B). Preparation C contained 10 mg of endotoxin-free KLH protein (keyhole limpet hemocyanin—Calbiochem) or polyclonal human IgG (Sigma) dissolved in 3.5 mL of PBS buffer. Formulations with as much as 90% w/w protein to total powder can be obtained by this procedure. One gram of preparation A+B was added to preparation C. The combined feed preparation was spray dried with a standard B-191 Mini spray drier equipped with a modified high efficiency cyclone under the following conditions: inlet temperature=75° C.; outlet temperature=46° C.; aspirator=89%; pump=2.2 mL/min; and, nitrogen flow=2400 L/h. The theoretical final composition was: 31.2% DPPC; 30% lactose; 30% protein; 7.8% calcium chloride dehydrate; and, 1% tyloxapol.

### EXAMPLE 2

### Electron Microscopy Data on Spray Dried Formulations of Antigens

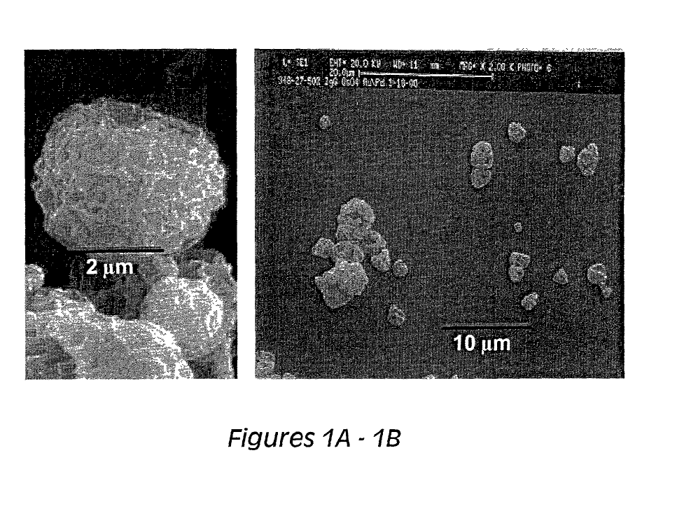

Formulations containing human IgG obtained as described in Example 1, were dehydrated, fixed and subjected to surface electron microscopy (SEM). The results, shown in FIG. 1, demonstrate that the formulation is comprised of particles with an irregular surface and diameters of 3-4 μm. In the left panel of FIG. 1 (FIG. 1A), there is a high-resolution picture of one particle and in the right panel (FIG. 1B), there is a low resolution image of multiple particles subjected to SEM.

As used throughout the specification and claims, these particles are referred to as spray dried lipid microparticles (“SDLM”).

### EXAMPLE 3

### Physical Characterization of Spray Dried Particles Loaded With Protein Antigens

Andersen cascade impactor analysis was carried out, using a prototype protein/macromolecule (human IgG, Sigma) loaded into SDLM generated as described in the Example 1. An amount of formulation corresponding to approximately 100 μg of hIgG was loaded into the system. The cascade impactor discs were retrieved and the fractions were quantified by dissolving the recovered powder from each disc in normal saline, followed by ELISA assay.

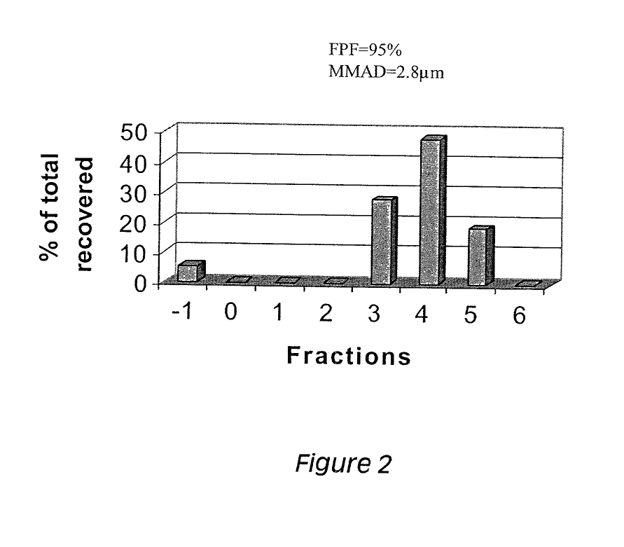

The assay was carried out by incubating supernatants onto microwells precoated with anti-human k+λ chain IgG monoclonal antibodies (Sigma Immunochemical) and blocked subsequently with SeaBlock (Pierce). Coating was carried out at 4° C. overnight with 500-fold diluted ascitic fluid. Blocking was carried out for 1 hour at 37° C. The samples were incubated for 2 hours at room temperature, in 10% SeaBlock dissolved in normal saline. After extensive washing, the assay was developed by one hour incubation with 1:1000 polyclonal goat anti-human IgG conjugated to alkaline phosphatase (Sigma Immunochemical), followed by addition of pNPP substrate (Sigma). The results were read using an automatic ELISA plate reader. The estimation was carried out by interpolation on a standard curve obtained with non-formulated IgG in normal saline. The results, shown in FIG. 2, are expressed as % of total amount recovered: the fractions 0-2 correspond to the upper respiratory tract; and 3-6 to the lower respiratory tract (bronchial and alveolar regions, respectively). Fraction (−1) corresponds to the delivery instrument. The fine particle fraction (FPF, deposited in the lower respiratory tract) was 95%. The mean mass aerodynamic diameter (MMAD) was 2.8 μm.

### EXAMPLE 4

### Enhancement of Th2 Cellular Responses to Foreign Antigen by Using Spray Dried Lipid Formulation

Even though the example is using a spray-dried formulation, it is anticipated that the manufacturing process will not greatly affect the enhancement of the Th2 response. The response is driven mainly by the composition and not by the manufacturing process. It is also contemplated that the particles (microparticles) can be obtained by homogenization (any type of colloidal suspension-emulsion, double emulsion, bicontinuous emulsions, micellar, isotropic solutions, etc), lyophilization, precipitation, solvent evaporation, flash evaporation—the preferred method is spray-drying.

Balb/c mice (2 month-old females from Harlan; n=4/group) were immunized on day 0 with SDLM (made according to Ex. 1) resuspended in perflubron (PFOB), by intraperitoneal (“i.p.”) injection. The volume of i.p. inoculum was 100 μl corresponding to 1,000 μg of formulation, which in turn contained 100 μg of antigen. Lower doses of SDLM were used in parallel. As a control, dose matched amount of keyhole lymphet hemocyanin “KLH” in normal saline was used.

The T cell response was estimated by ELISPOT analysis at 7 days after immunization, using nitrocellulose wells (Millipore) precoated with 4 μg/ml of anti-IFNγ or anti-IL-4 rat anti-mouse monoclonal antibodies (BD-PharMingen). Splenocytes were stimulated in vitro with mitomycin-treated syngeneic APC in the presence of 10 μg/ml KLH antigen. Generally, the assay was carried out by using serial dilutions of cell suspension, ranging from 4×10⁵ to 5×10⁴ responder cells/well. The number of stimulator cells was 2×10⁵ cells/well. After 72 hours, the assay was developed using sequential steps of washing, incubation with 2 μg/ml of biotinylated anti-cytokine antibodies (BD-PharMingen), streptavidin-HRP and insoluble substrate (AEC). The frequency of spot-forming-colonies (SFC) was measured using an automatic acquisition system equipped with Image-Pro Plus software.

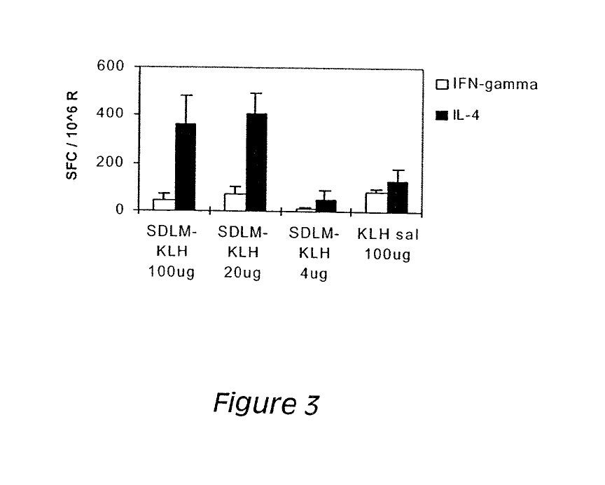

The results, as shown in FIG. 3, are expressed as number of spot forming colonies (SFC)/10⁶ splenocytes (mean ±SE of quadruplicates). The results show enhanced IL-4 responses were triggered by the formulated KLH antigen. As shown in FIG. 3, KLH-SDLM formulated with both 100 μg and 20 μg of KLH produced a significantly greater IL-4 response than 100 μg of KLH in normal saline (control).

### EXAMPLE 5

### Slow Release Effect is Responsible for the Enhancement of Th2 Response in Context of Spray Dried Formulations. Repeated, Low Doses of Non-Formulated KLH Triggered Enhanced Th2 Responses

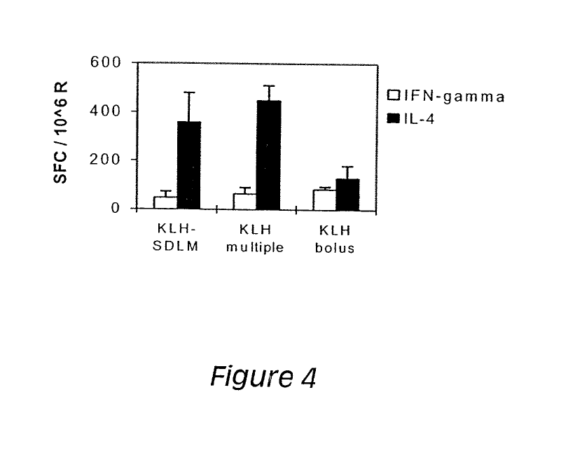

Balb/c mice (2 month-old females from Harlan; n=4/group) were immunized on day 0 with SDLM (made according to Ex. 1) resuspended in perfluorooctyl bromide (“perflubron” or “PFOB”), by intraperitoneal (“i.p.”) injection (“KLH SDLM”; see FIG. 4). The volume of intraperitoneal inoculum was 100 μl corresponding to 1,000 μg of formulation, which in turn contained 100 μg of antigen. As a control, dose matched amount of KLH in normal saline was used (100 μg of hIgG, “KLH bolus”; see FIG. 4). In parallel, non-formulated KLH was administered 3 times (day 0, 3, 6) at doses of 33 μg/injection (“KLH multiple”; see FIG. 4).

The T cell response was estimated by ELISPOT analysis at 7 days, using nitrocellulose wells (Millipore) precoated with anti-IFNγ or anti-IL-4 antibodies, as described in Example 4. After 72 hours, the assay was developed using sequential steps of washing, incubation with anti-cytokine antibodies, streptavidin-HRP and insoluble substrate (AEC). The frequency of spot-forming-colonies (SFC) was measured using an automatic acquisition system controlled by Image-Pro Plus software. The data is expressed, as shown in FIG. 4, as frequency of SFC associated with cytokine production (mean ±SE). FIG. 4 demonstrates that slow release is responsible for the enhancement of Th2 response. FIG. 4 shows a significant enhancement of IL-4 by KLH-SDLM approximating the IL-4 production of non-formulated KLH administered separately on days 0, 3 and 6 and significantly greater enhancement of IL-4 than the control (“KLH-Bolus”).

### EXAMPLE 6

### Enhancement of Th2-Dependent IgG1 Response Against Antigen Protein Formulated into Spray Dried Microparticles

Balb/c mice (2 month-old females from Harlan; n=4/group) were immunized on day 0 with SDLM (made according to Example 1) resuspended in perflubron (PFOB), by intraperitoneal injection (“KLH SDLM”). The volume of intraperitoneal inoculum was 100 μl corresponding to 1,000 μg of formulation, which in turn contained 100 μg of antigen. Three different 10% KLH formulations were tested: (1) HES-SDLM containing 10% hydroxyethylstarch (“HES”); (2) Lac-Ca-SDLM containing 10% lactose (“Lac”) and Ca²⁺ in equimolar amounts with DPPC; and, (3) Lac-SDLM containing 10% lactose. As a control, dose matched amount of KLH in normal saline was used (100 μg of KLH, bolus).

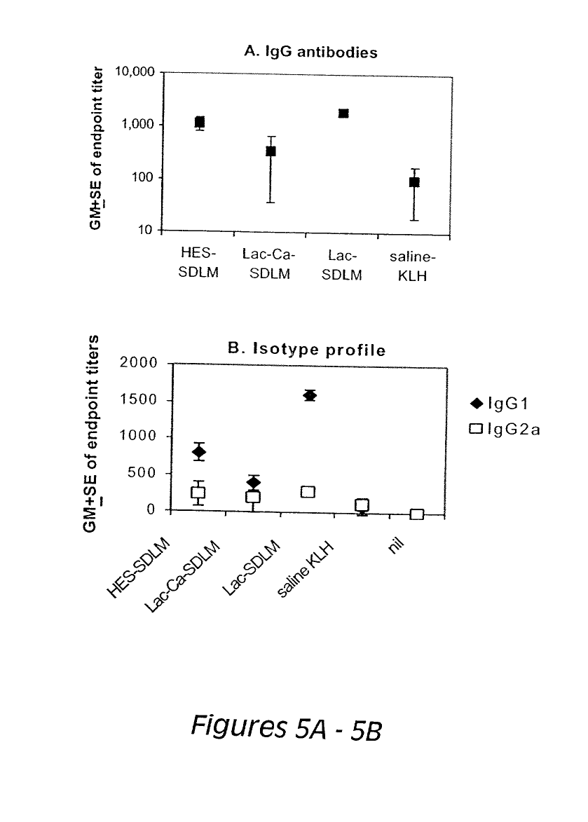

At day 7 after vaccination, blood samples were harvested from the mice and KLH-specific IgG was measured by ELISA. In addition, the presence of specific IgG1 and IgG2a antibodies was assessed. The ELISA plates were pre-coated with 10 μg/ml KLH, blocked with 30% SeaBlock and incubated with 2-fold dilutions of serum samples for 2 hours at room temperature. As developing reagents, 1 hour incubation times were used at room temperature with 1:1000 polyclonal goat anti-mouse IgG antibodies coupled with alkaline phosphatase (Sigma Immunochemical), or 1:250 biotinylated rat anti-mouse IgG1 and anti-IgG2a antibodies (Biosource International). The reaction was developed using an additional streptavidin-alkaline phosphatase conjugate step (dilution 1:1000, for 1 hour at room temperature) in case of biotinylated antibodies, followed by addition of pNPP substrate (Sigma). The assay was read using an automated plate reader. The results are expressed as geometric means of endpoint titers ±SE as shown in FIGS. 5A and 5B (FIG. 5A: whole IgG and FIG. 5B: IgG2a and IgG1). FIG. 5A demonstrates that increase or decrease of immune response varies and may be controlled based on excipients used. FIG. 5B shows increased titers of KLH specific to IgG1 antibodies but not IgG2a antibodies and dependency of antibody titers on coexcipients.

### EXAMPLE 7

### Enhancement of IgG1 Antibody Response to Formulated Antigen Depends on CD3⁺ T Cells

CD3ε deficient or C57BL/6 wild-type (“wt”) counterparts (2 month-old females from The Jackson Labs; n=4/group) were immunized on day 0 with SDLM (made according to Ex. 1) resuspended in perflubron (PFOB), by intraperitoneal injection (“SDLM KLH”). The volume of intraperitoneal (“i.p.”) inoculum was 100 μl corresponding to 1,000 μg of formulation, which in turn contained 100 μg of antigen. As a control, dose matched amount of KLH in normal saline was used (100 μg of antigen).

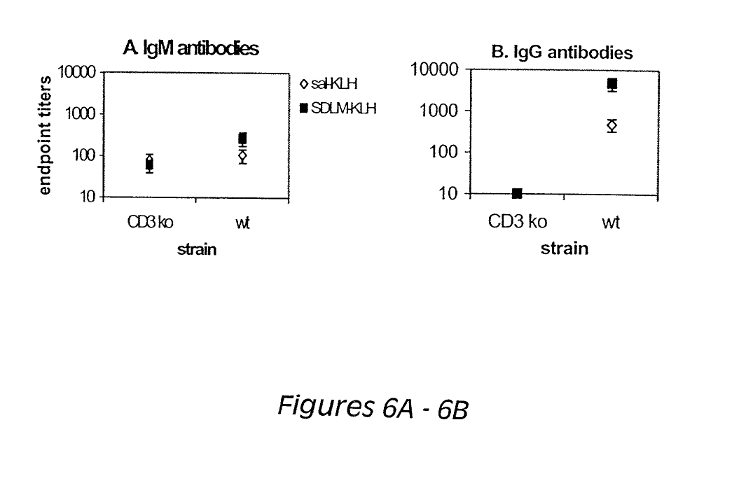

At day 7 after vaccination, blood samples were harvested from the mice and KLH-specific IgM (FIG. 6A) and IgG antibodies (FIG. 6B) were measured by ELISA. The ELISA plates were pre-coated with 10 μg/ml KLH, blocked with 30% SeaBlock and incubated with 2-fold dilutions of serum samples for 2 hours at room temperature. As developing reagents, 1 hour incubation times at room temperature were used with 1:1000 polyclonal goat anti-mouse IgM or IgG antibodies coupled with alkaline phosphatase (Sigma Immunochemical). Finally, pNPP substrate (Sigma) was added and the assay was read using an automated plate reader. The results are expressed as means ±SE of endpoint titers and are shown in FIGS. 6A-6B. FIGS. 6A-6B show that enhancement of IgG1 antibody response to formulated antigen depends on CD3⁺ cells (see FIG. 6B) and in CD3ε defective mice, the enhancement of IgG1 response to KLH is impaired, demonstrating that IgG1 is assisted by CD3⁺ T cells which are a critical requirement for TH₂ immunity.

### EXAMPLE 8

### Enhancement of Th2 Response in Context of Spray-Dried Lipid Formulations Requires the Expression of MHC Class II Molecules

MHC class II deficient (Abb−/−) mice or C57BL/6 wild-type (“wt”) counterparts (2 month-old females from The Jackson Labs; n=3/group) were immunized on day 0 with SDLM (as made in Ex. 1) resuspended in perflubron (PFOB), by intraperitoneal injection (“SDLM KLH”). The volume of i.p. inoculum was 100 μl corresponding to 1,000 μg of formulation, which in turn contained 100 μg of antigen. As a control, dose matched amount of KLH in normal saline was used (100 μg of antigen).

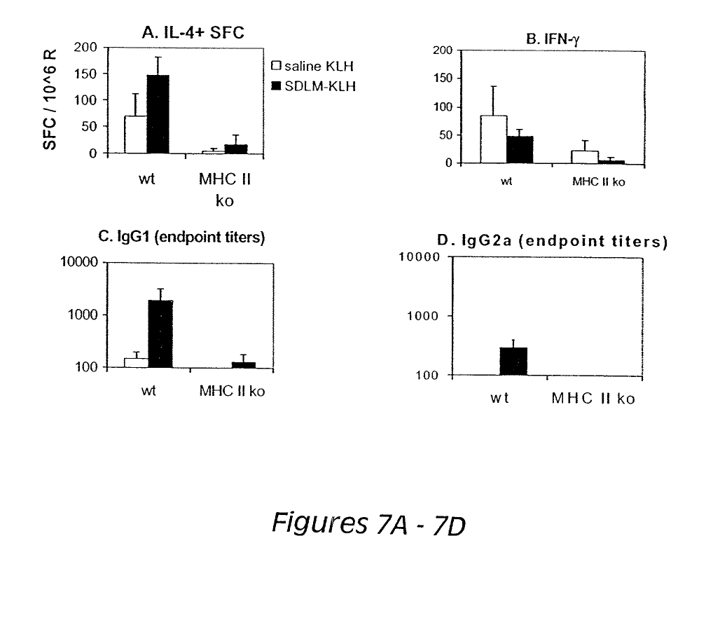

The T cell response was estimated by ELISPOT analysis at day 7 after immunization, using nitrocellulose wells (Millipore) precoated with anti-IFNγ or anti-IL-4 antibodies (BD-PharMingen). Splenocytes were stimulated in vitro with mitomycin-treated syngeneic APC in the presence of 10 μg/ml KLH antigen. Generally, the assay was carried out by using serial dilutions of cell suspension, ranging from 4×10⁵ to 5×10⁴ responder cells/well. The number of stimulator cells was 2×10⁵ cells/well. After 72 hours, the assay was developed using sequential steps of washing, incubation with biotinylated anti-cytokine antibodies, streptavidin-HRP and insoluble substrate (AEC). The frequency of spot-forming-colonies (SFC) was measured using an automatic acquisition system controlled by Image-Pro Plus software. The results are expressed as SFC /10⁶ responder cells (FIGS. 7A-7B).

At day 7 after vaccination, blood samples were harvested and KLH-specific IgG1 and IgG2a were measured by ELISA. The ELISA plates were pre-coated with 10 μg/ml KLH, blocked with 30% SeaBlock and incubated with 2-fold dilutions of serum samples for 2 hours at room temperature. As developing reagents, 1 hour incubation times at room temperature were used with 1:250 biotinylated rat anti-mouse IgG1 and anti-IgG2a antibodies (Biosource International). The reaction was developed using an additional streptavidin-alkaline phosphatase conjugate step (dilution 1:1000, for 1 hour at room temperature) followed by addition of pNPP substrate (Sigma). The assay was read using an automated plate reader. The results in FIGS. 7A-7D are expressed as geometric means of endpoint titers ±SE (FIG. 7C: IgG1 and FIG. 7D: IgG2a) and show that in the absence of MHC II expression, Th₂ response is lost and subsequently there is impaired antibody response.

### EXAMPLE 9

### Increase of B Cell Response to Formulated Antigens in the Absence of Functional CD3 Molecules

CD3ε deficient or C57BL/6 wild-type (“wt”) counterparts (2 month-old females from The Jackson Labs; n=4/group) were immunized on day 0 with SDLM (as made in Ex. 1) resuspended in perflubron (PFOB), by intraperitoneal injection (closed bars). The volume of i.p. inoculum was 100 μl corresponding to 1,000 μg of formulation, which in turn contained 100 μg of antigen. As a control, dose matched amount of KLH in normal saline was used (100 μg of antigen; open bars).

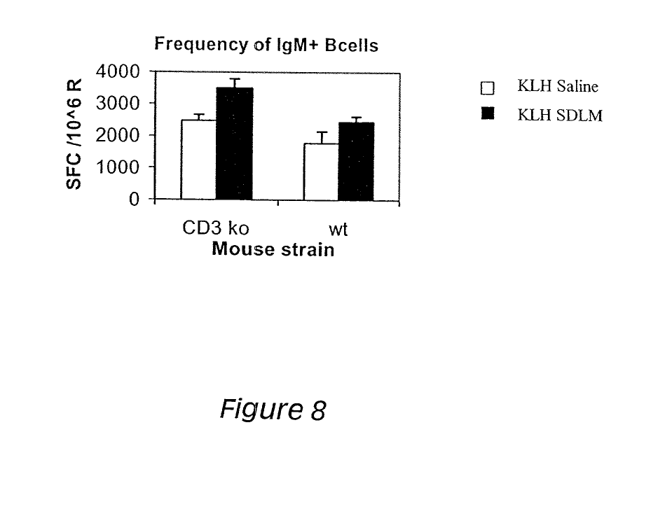

At 7 days spleens were harvested and a modified ELISPOT assay was carried out: the cells were stimulated with plate-bound KLH for 72 hours at a concentration of 5×10⁶/ml and the assay was developed by incubation with 2 μg/ml of biotinylated anti-IgM antibody (BD-PharMingen), followed by streptavidin-horseradish peroxidase (Biosource Int.) and AEC substrate (Sigma). The number of IgM⁺ SFC was read using an automatic system equipped with Image-Pro software. The results are expressed in FIG. 8 as number (means ±SEM) of SFC/10⁶ splenocytes and shows expression of antigen specific B cells in the absence of functional T cells, supporting a direct effect of SDLM formulation on antigen state. Both the CD3 knock-out (“CD3 ko”) and wild type (“w.t.”) spleen cells showed increased IgM+B cell expansion subsequent to administration of KLH formulated into microparticles over the saline KLH control.

### EXAMPLE 10

### Enhanced Loading With Antigen of Antigen Presenting Cells (APC) by Use of Spray-Dried Lipid Formulations

CD3ε deficient or C57BL/6 wild-type (“wt”) counterparts (2 month-old females from The Jackson Labs; n=4/group) were immunized on day 0 with SDLM (made according to Ex. 1) resuspended in perflubron (PFOB), by intraperitoneal injection. The volume of i.p. inoculum was 100 μl corresponding to 1,000 μg of formulation, which in turn contained 100 μg of antigen. As a control, dose matched amount of KLH in normal saline was used to immunize CD3ε deficient mice (100 μg of antigen). APC were used from CD36 deficient mice since there was no possibility of contamination with endogenous T cells; the responder cells were generated from T cell competent, C57BL/6 mice.

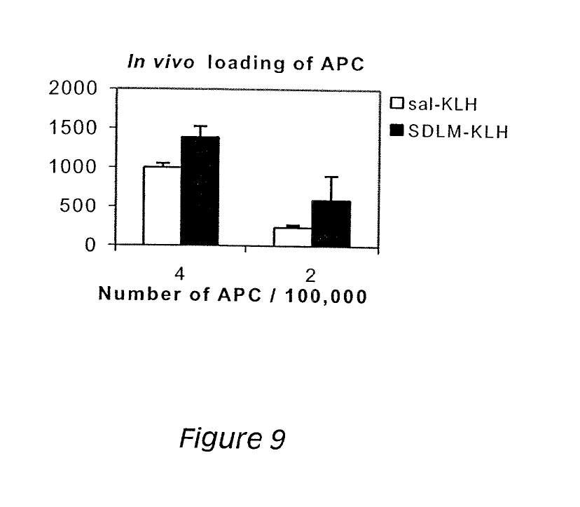

The T cell response was estimated by ELISPOT analysis, using nitrocellulose wells (Millipore) precoated with 4 μg/ml of anti-IL-4 antibodies (BD-PharMingen). Memory T cells from mice immunized with KLH in complete Freund's adjuvant, were stimulated in vitro (10⁵ cells/well) with mitomycin-treated syngeneic APC harvested from mice injected 7 days prior to evaluation, with KLH in saline (“KLH-sal”) or formulated antigen (“SDLM-KLH”). The number of stimulator cells was 2 or 4×10⁵ cells/well. After 72 hours, the assay was developed using sequential steps of washing, incubation with 2 μg/ml anti-cytokine antibody overnight at 4° C., followed by incubation with 1:1000 streptavidin-HRP (Biosource Int.) and insoluble substrate (AEC). The frequency of spot-forming-colonies (SFC) was measured using an automatic acquisition system controlled by Image-Pro Plus software. The results are expressed as IL-4⁺ SFC/well (means ±SEM of triplicates) as shown in FIG. 9 and demonstrate enhanced ability of APC purified from mice, injected with formulated antigen, to stimulate memory T cells. FIG. 9 shows that APC separated from SDLM-treated animals are superior in the ability to stimulate antigen-specific T cells, compared to APC separated from saline treated mice.

### EXAMPLE 11

### Aggregated Nature of Protein Upon Formulation into Spray Dried Particles

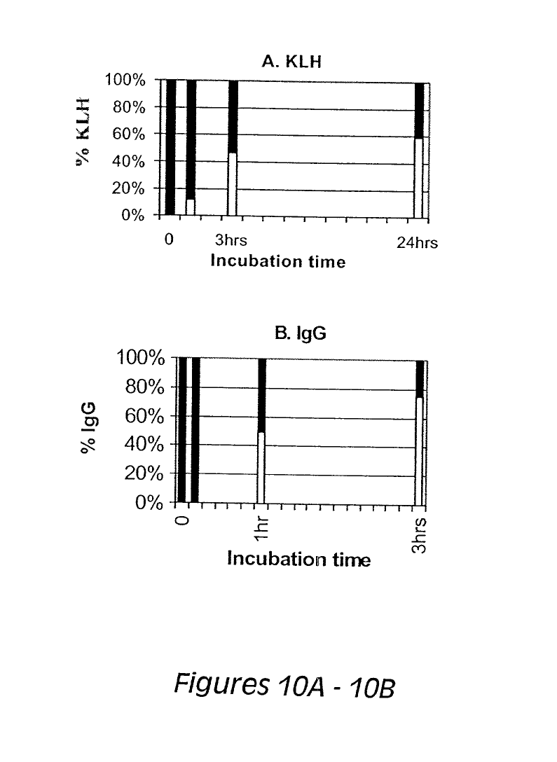

SDLM (made according to Ex. 1) loaded with KLH were incubated in saline (1 mg SDLM/ml of PBS) at 37° C. under mild shaking conditions (60 cycles/minute). At various intervals, samples were harvested and ultracentrifuged (5 minutes at 10,000 RPM). The protein was completely released from pellet by using detergent buffer (0.1% SDS-PBS). The concentration of protein in supernatant and pellet was measured using the biuret reaction (BioRad Laboratories) in case of KLH (FIG. 10A) or by capture ELISA in case of IgG like in Example 3 (FIG. 10B) and expressed as percentage of the total amount. As shown in FIGS. 10A-10B, the percentage of protein associated with the pellet (aggregated) is represented with closed bars. With open bars, the percentage of protein in supernatant is represented. In parallel, it is demonstrated that non-formulated KLH and IgG segregate with aqueous supernatant. FIGS. 10A-10B show that formulation into SDLM is a useful method to aggregate immunoglobulins and other proteins with matrix excipients.

### EXAMPLE 12

### Enhanced Th2 Cellular Responses to Antigenic Immunoglobulins Formulated into Spray Dried Microparticles Results in Modulation of Immune Response Upon Repeated [in vivo] Delivery Enhanced Th2 Responses

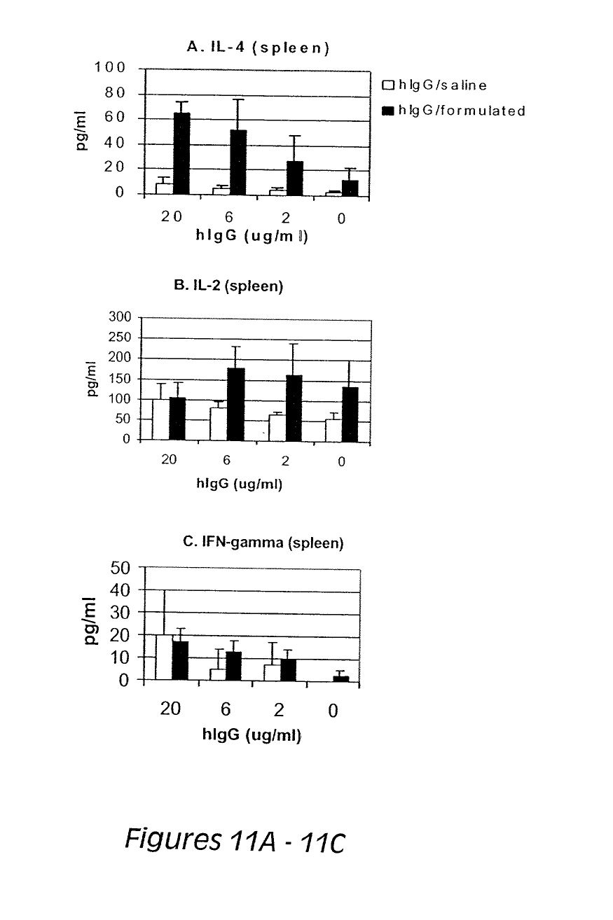

BALB/c mice (2 month-old females from Harlan Sprague) were immunized three times with antigenic human IgG (Sigma) in saline (open bars) or formulated into SDLM (made according to Ex. 1) (closed bars) by nasal instillation (40 μl of suspension corresponding to 40 μg of hIgG/dose). The schedule of immunization was: week 0, week 2 and week 4. At 6 weeks, the animals (3/group) were sacrificed and spleens harvested. The T cell response in the spleen was evaluated by ELISA using kits purchased from BioSource International. Splenocytes were stimulated in vitro with mitomycin-treated syngeneic APC in the presence of 10 μg/ml hIgG. The assay was carried out by using 5×10⁵ responder cells/well. The number of stimulator cells was 2×10⁵ cells/well. After 72 hours, supernatants were harvested and the concentration of cytokine estimated by ELISA, using plates precoated with anti-cytokine antibodies and developing reagents from BioSource. The results are expressed in FIGS. 11A-11C as mean ±SD of triplicates (pg/ml cytokine in cell culture supernatant). FIGS. 11A-11C show that repeated administration of formulated human IgG into mice result results in expansion of IL-4 producing T cell population. For example, FIG. 11A shows significant increase in IL-4 production from administration of formulated human IgG (hIgG/formulated) in mice over the control (hIgG/saline).

### EXAMPLE 13

### Enhanced Th2-Dependent IgG1 Responses to Antigenic Immunoglobulin Formulated in Spray-Dried Microparticles

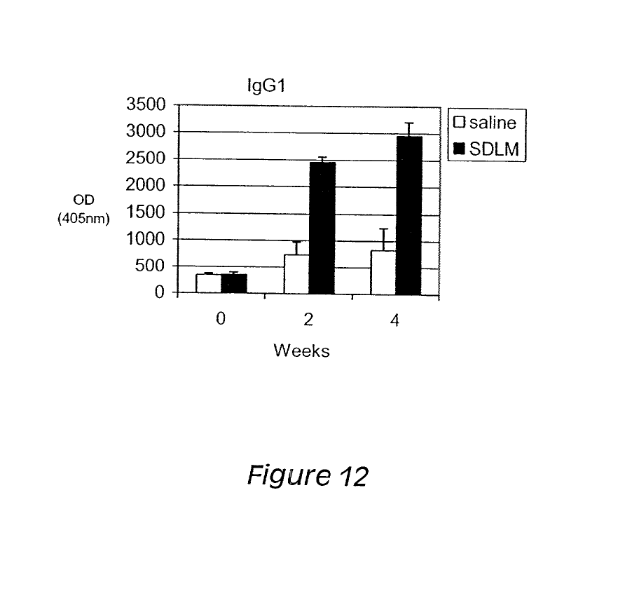

BALB/c mice (2 month-old females from Harlan Sprague; n=4/group) were immunized with antigenic human IgG (Sigma) in saline (open bars) or formulated into SDLM (closed bars) by intraperitoneal injection (100 μl of suspension corresponding to 100 μg of hIgG/dose). At various intervals after immunization, blood samples were harvested and the IgG1 antibodies measured using ELISA assay. The ELISA plates were pre-coated with 10 μg/ml hIgG, blocked with 30% SeaBlock and incubated with 1:50 dilution of serum samples for 2 hours at room temperature. As developing reagents, 1 hour incubation times at room temperature were used with 1:250 biotinylated rat anti-mouse IgG1 antibody (Biosource International). The reaction was developed using an additional streptavidin-alkaline phosphatase conjugate step (dilution 1:1000, for 1 hour at room temperature) followed by addition of pNPP substrate (Sigma). The assay was read using an automated plate reader. The results are expressed as mean +SD of OD at 405 nm and are shown in FIG. 12 and show significant IgG1 antibody production with formulated SDLM over the saline control.

### EXAMPLE 14

### Spray Dried Lipid Based Composition of Immunoglobulin Recombinant Constructs Bearing an Engrafted Epitope Peptide

For the preparation of SDLM containing immunoglobulin recombinant constructs, a spray drying technology was used as follows. Preparation A was comprised of a liposome suspension of 0.70 g of DPPC dispersed in 23 g of hot DI water with a T-25 Ultraturrax at 9000 rpm for about 5 min. The coarse liposomes were homogenized under high pressure (18,000 psi) for 5 discrete passes with an Avestin Emulsiflex C5. Preparation B contained 0.18 g of CaCl₂.2H₂O, 0.012 g of tyloxapol and 0.18 g of lactose monohydrate. Preparation A was added i to dissolve all the ingredients in preparation B, now called preparation (A+B). Preparation C contained 10 mg of immunoglobulin construct bearing an engrafted T cell epitope peptide (IgHA, composed of mouse IgG2b anti-arsonate antibody engrafted with the I-E^{d}-restricted hemagglutinin epitope of influenza virus: SFERFEIFPKE) dissolved in 10 mL of PBS buffer. Two grams of preparation A+B was added to preparation C. The combined feed preparation was spray dried with a standard B-191 Mini spray drier equipped with a modified high efficiency cyclone under the following conditions: inlet temperature=80° C.; outlet temperature=47° C.; aspirator=83%; pump=2.2 mL/min; and, nitrogen flow=2400 L/h.

The theoretical final composition was: 59.1% DPPC; 15% Lactose; 10% IgHA; 15% calcium chloride dihydrate; and, 1% tyloxapol.

### EXAMPLE 15

### Immunogenicity of Immunoglobulin Construct Bearing an Engrafted T Cell Epitope

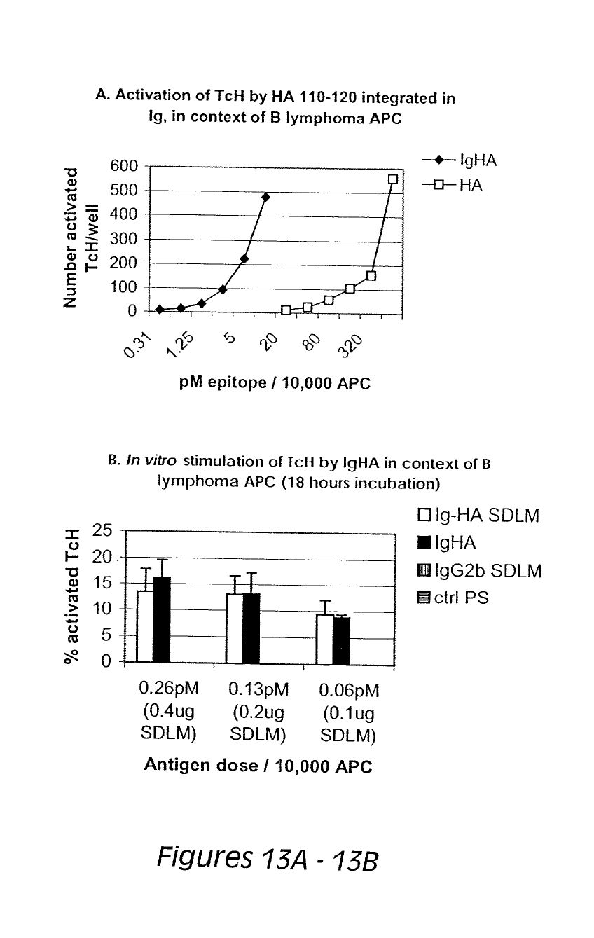

In a preliminary analysis, the ability of HA peptide (sequence SFERFEIFPKE) and Ig-HA construct (SFERFEIFPKE engrafted into CDR3 region of mouse IgG2b) to stimulate specific T cell hybridoma (TcH) was assessed (FIG. 13A). The TcH was engineered to express a standard β-galactosidase gene under the control of IL-2 promoter. A constant number of APC (10⁴ M12 B cell lymphoma cells) were incubated with 2×10⁴ TcH in the presence of various amounts of HA or IgHA. After 24 hours of incubation at 37° C. and 5% CO₂, the cells were washed in wells with PBS, fixed with glutaraldehyde+formaldehyde and washed again with saline. The reaction was developed using a saturated solution of X-gal substrate. The results were obtained by microscopy and expressed as number of activated (blue) TcH/well at various concentrations of HA and IgHA as shown in FIG. 13A.

A similar protocol was pursued in case of IgHA formulated in SDLM (Ig-HA SDLM; FIG. 13B)(made according to Ex. 14). Microparticles were dried on plastic wells, before addition of APC and TcH. In parallel, non-formulated IgHA, control IgG2b devoid of HA insert (“IgG2b SDLM”) and particles devoid of protein (“PS”) were also used. The results are expressed as % activated TcH at various concentrations of epitope, corresponding to different amounts of formulation as shown in FIG. 13B. FIG. 13B shows in vitro stimulation of TCH by IgHA SDLM in context of B lymphoma APC (18 hours incubation) and shows that IG-HA formulated into SDLM maintained its immunogenicity equal to the nonformulated IgHA (note that IgG2b SDLM and control PS produced no stimulation of TCH).

### EXAMPLE 16

### Spray Dried Lipid Based Composition of Self Antigens Encompassing Disease-Associated Epitopes

Preparation A was comprised of a liposome suspension of 0.70 g of DPPC dispersed in 23 g of hot DI water with a T-25 Ultraturrax at 9000 rpm for about 5 min. The coarse liposomes were homogenized under high pressure (18,000 psi) for 5 discrete passes with an Avestin Emulsiflex C5. Preparation B contained 0.18 g of CaCl₂.2H₂O, 0.012 g of tyloxapol and 0.1 g of hydroxyethylstarch (HES, Ajinomoto, Japan). Preparation A was added to dissolve all the ingredients in preparation B, now called preparation (A+B). Preparation C contained 25 mg of bovine insulin B chain (Sigma) dissolved in 10 mL of PBS buffer. Two grams of preparation A+B was added to preparation C. The combined feed preparation was spray dried with a standard B-191 Mini spray drier equipped with a modified high efficiency cyclone under the following conditions: inlet temperature=80° C.; outlet temperature=47° C.; aspirator=83%; pump=2.2 mL/min; and, nitrogen flow=2400 L/h. The theoretical final composition was: 49% DPPC; 10% HES; 25% InsB; 15% calcium chloride dehydrate; and, 1% tyloxapol.

### EXAMPLE 17

### Modification of Ongoing Autoreactive Immune Response by Administration of Self-Antigen Formulated in Spray Dried Particles

Mice were treated at the age of 4, 5 and 6 weeks with 25%-InsB-SDLM (made according to Ex. 16) delivered by nasal instillation under mild METOFANE anesthesia, at a dose of 400 μg of formulation in 40 μl of PFOB, corresponding to 100 μg of antigen. As controls, naïve female NOD mice were used, or mice treated with non-formulated insulin B in saline, or injected subcutaneously with insulin B emulsified in incomplete Freund's adjuvant (IFA) at the age of 4, 5 and 6 weeks. Spleens were harvested at the age of 30-31 weeks in the case of mice that did not progress to diabetes. Single cell suspension was prepared from each spleen (n=4/group) and after hypotonic lysis of red blood cells, the splenocytes were resuspended in HL-1 medium (BioWhittaker).

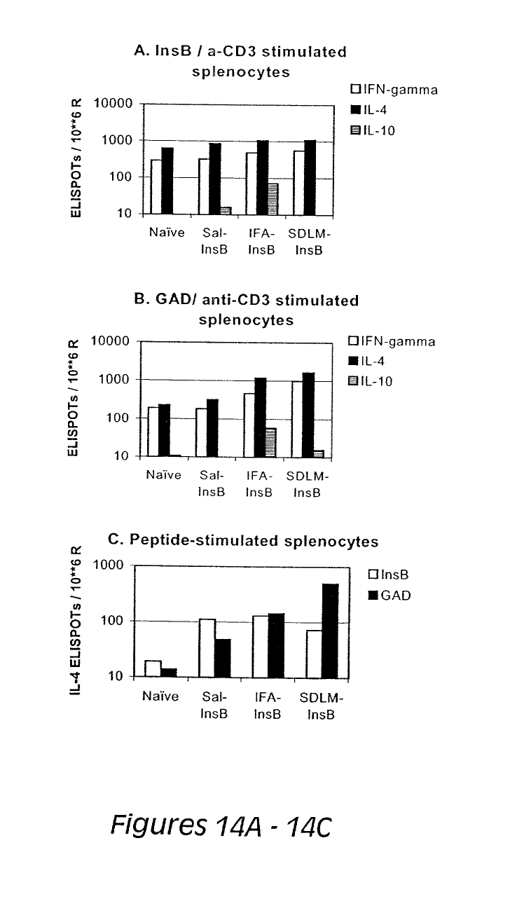

The splenocytes were incubated for 5 days with bovine oxidized insulin B (InsB) or a mixture of GAD-65-derived peptides (Research Genetics) (5×10⁶ cells/3 ml, in the presence of 20 μg/ml of peptide). After the preliminary incubation with peptides, the T cells were expanded by replacing the cell culture medium with fresh HL-1 medium alone (FIG. 14C) or supplemented with a mixture of 5 μg/ml of anti-CD3 mAb +2 μg/ml of anti-CD28 mAb (PharMingen) (FIGS. 14A and 14B). The stimulation was carried out in ELISPOT plates precoated with 4 μg/ml of anti-cytokine antibodies (PharMingen). The ELISPOT assay was developed by overnight incubation at 4° C. with 2 μg/ml of biotinylated anti-cytokine antibodies (PharMingen), followed by addition of streptavidin/horseradish peroxidase conjugate (BioSource) and insoluble substrate (AEC, Sigma).

The data was acquired and processed using a CDC camera connected to a computer operating ImagePro 4.1 image analysis software. The results are expressed in FIGS. 14A-14C as number of spots (“ELISPOTs”)/10⁶ responder cells. FIGS. 14A-14C shows a modified splenic profile (expansion of cytokine producing population) of T cells reactive against disease-associated peptides, in non-obese diabetic animals treated with spray dried particles loaded with insulin B chain. FIG. 14C shows significant expression of IL-4 with SDLM—Ins B over the control Sal-InsB.

### EXAMPLE 18

### Modification of Ongoing Autoreactive Immune Response by Administration of Self Antigen Formulated in Spray Dried Particles

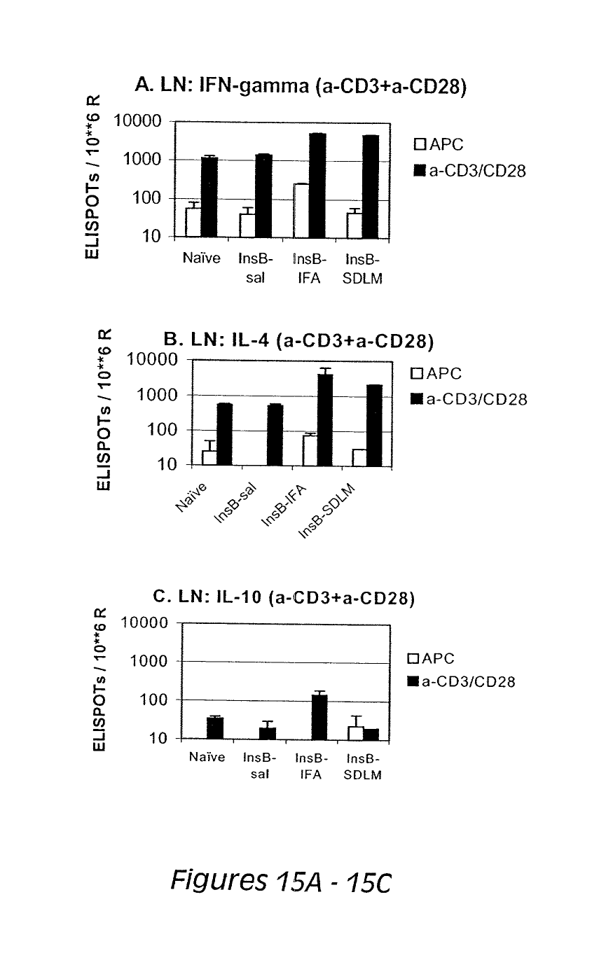

Mice were treated at the age of 4, 5 and 6 weeks with 25%-InsB-SDLM (made according to Ex. 16) delivered by nasal instillation under mild METOFANE anesthesia, at a dose of 400 μg of formulation in 40 μl of PFOB, corresponding to 100 μg of antigen. As controls, naïve female NOD mice were used, or mice treated intranasally with dose-matched non-formulated insulin B in saline, or injected subcutaneously with insulin B emulsified in incomplete Freund's adjuvant (IFA) at the age of 4, 5 and 6 weeks. Pancreatic lymph nodes were harvested at the age of 30 weeks from non-diabetic animals and placed immediately in ice-cold DMEM medium. The lymph nodes were pooled from 3-4 animals/group. Subsequently, they were incubated in DMEM-0.45% BSA, supplemented with collagenase, for 1 hour at 37° C. Single cell suspension was obtained by passing through Falcon strainers and the cells were incubated in ELISPOT plates previously coated with 4 μg/ml of anti-IFN-γ (FIG. 15A), anti-IL-4 (FIG. 15B) or anti-IL-10 (FIG. 15C) antibodies (PharMingen), at concentrations of 2, 1, 0.5 and 0.25×10⁵ cells/100 μl in HL-1 medium and in the presence of 5 μg/ml of anti-CD3 mAb+2 μg/ml of anti-CD28 mAb (PharMingen). Alternatively, the lymph node cells were incubated only with mitomycin-treated splenocytes (2×10⁵ cells/well). After 48 hours of incubation under standard cell culture conditions, the cells were washed off and the assay was read by incubation with 2 μg/ml of biotinylated anti-cytokine antibodies (PharMingen) and subsequently, 1:1000 streptavidin-horseradish peroxidase and insoluble substrate (AEC, Sigma). The results are expressed in FIGS. 15A-15C as means ±SE of number of cytokine-producing cells normalized to 10⁶ responder cells. FIGS. 15A-15C show a modified lymph node profile of T cells (expansion of cytokine producing T cells), in non-obese diabetic animals treated with spray dried particles loaded with insulin B chain. FIG. 15B in particular shows significant IL-4 production over the control of cells from mice treated with InsB-SDLM.

### EXAMPLE 19

### Modification of Ongoing Autoreactive Immune Response by Administration of Self Antigen Formulated in Spray Dried Particles

Mice were treated at the age of 4, 5 and 6 weeks with 25%-InsB-SDLM (made according to Ex. 16) delivered by nasal instillation under mild METOFANE anesthesia, at a dose of 400 μg of formulation in 40 μl of PFOB, corresponding to 100 μg of antigen. As controls, naïve female NOD mice were used, or mice treated intranasally with dose-matched non-formulated insulin B in saline, or injected subcutaneously with insulin B emulsified in incomplete Freund's adjuvant (IFA) at the age of 4, 5 and 6 weeks. Whole pancreas was removed from 3-4 mice/treatment group at the age of 30 weeks and immediately placed in ice-cold DMEM. The pancreas tissue was sliced and digested with collagenase in a Ca²⁺ Mg²⁺-free buffer for 45 minutes at 37° C. under mild shaking conditions. The digestion was stopped when the released islets were visible by inverted microscopy, by washing with ice-cold HL-1 medium followed by centrifugation. The resulting pellet was passed through 70 μm cell strainers using a 3 ml syringe piston and washed with 4° C.-cold RPMI. The pellet was suspended in 2 ml of HL-1 medium, divided into two wells for each pancreas and incubated overnight in standard cell culture conditions. The wells were previously coated with anti-mouse CD3 antibody (10 μg/ml; PharMingen) and the incubation of cells was carried out in the presence of 2 μg/ml of anti-CD28 mAb (PharMingen). The concentration of cytokines in the supernatant was measured using ELISA kits (BioSource Int.) and expressed as means ±SE (pg/ml).

Tables 1A-1B show a modified profile in pancreas (decreased pro-inflammatory cytokines) in non-obese diabetic animals treated with spray dried particles loaded with insulin B chain.

TABLE 1A
Cytokine production at the age of 30-31 weeks:

| Group | IL-4 | IL-10 | TGF-β1 | IFN-γ | IL-1β |
|---|---|---|---|---|---|
| Naïve | 0 | 4 ± 1 | 294 ± 44 | 50 ± 20 | 20 ± 7 |
| SDLM-HES | 0 | 3 ± 1 | 361 ± 41 | 22 ± 20 | 37 ± 32 |
| InsB-sal | 0 | 2 ± 1 | 565 ± 101 | 2 ± 2 | 32 ± 10 |
| InsB-IFA | 0 | 3 ± 2 | 345 ± 71 | 0 | 10 ± 3 |
| SDLM-HES-InsB | 0 | 3 ± 1 | 310 ± 39 | 0 | 10 ± 2 |

TABLE 1B
Cytokine production at 11-12 weeks:

| | IFN-γ | IL-1β | IL-4 | IL-10 | TGF-β1 |
|---|---|---|---|---|---|
| Naïve | 74 ± 16 | 135 ± 61 | 0 | 7 ± 2 | 64 ± 21 |
| SDLM-HES-InsB | 75 ± 6 | 0 | 0 | 5 ± 2 | 129 ± 29 |

### EXAMPLE 20

### Suppression of Autoimmune Diabetes by Administration of Self Antigen Formulated in Spray Dried Particles

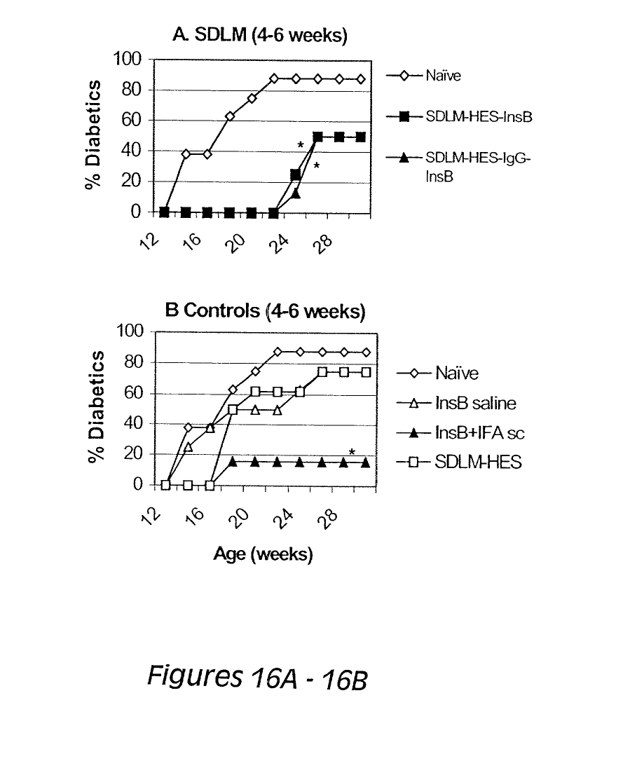

Female NOD mice were anesthetized with METOFANE and dosed via nostrils (weekly at the age of 4, 5 and 6 weeks) with SDLM (400 μg/40 μl in perflubron-Liquivent®) containing 10% HES and 25% of insulin B chain (InsB) (made according to Ex. 16). In parallel, a group of mice was treated with SDLM loaded with InsB and 10% of mouse IgG (Sigma Immunochemicals). The kinetics of diabetes was determined by measuring blood glucose levels weekly, starting with the age of 12 weeks. Mice with blood glucose levels in excess of 300 mg/dl were considered diabetic. The variation in kinetics of onset of disease between treated groups and naïve controls (FIG. 16A) was statistically significant (*p log-rank test <0.001).

Control groups (FIG. 16B) were observed in parallel: mice were instilled (3 times, at 4, 5 and 6 weeks) with similar amounts of SDLM containing 10% of HES devoid of self antigen, dose-matched non-formulated insulin B chain or injected (subcutaneously, at the age of 4, 5 and 6 weeks) with dose-matched InsB emulsified in IFA. Only injection of InsB+IFA resulted in significant suppression of disease (p log-rank test <0.001). FIGS. 16A and 16B show the kinetics of disease in non-obese diabetic mice treated with spray dried particles loaded with insulin B chain. FIG. 16A shows a significant reduction in the percentage of diabetic mice treated with either SDLM-HES-InsB or SDLM-HES-IgG-Ins-B.

### EXAMPLE 21

### Suppression of Autoimmune Diabetes by Administration of Self Antigen Formulated in Spray Dried Particles: Lack of Dependency on Endogenous IL-4 Production

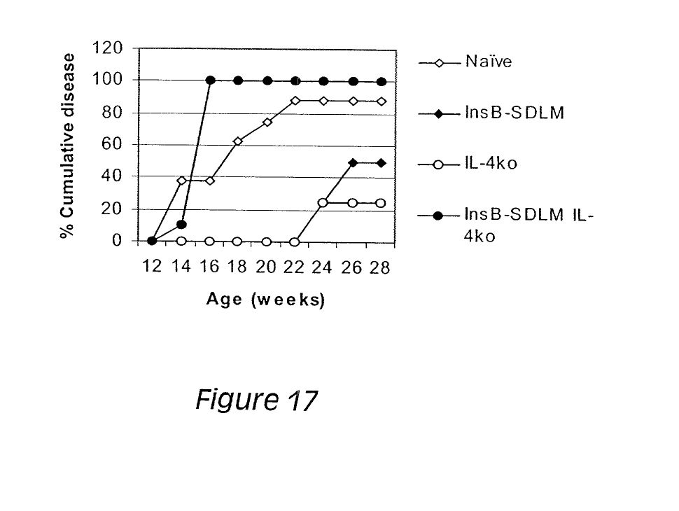

Female NOD mice (wild type or IL-4 deficient, purchased from The Jackson Labs) were anesthetized with METOFANE and dosed via nostrils (weekly at the age of 4, 5 and 6 weeks) with SDLM (made according to Ex. 16) 400 μg/40 μl in perflubron-Liquivent®) containing 10% HES and 25% of insulin B chain (InsB). As controls, naive animals were used. The kinetics of diabetes was determined by measuring blood glucose levels weekly, starting with the age of 12 weeks. Mice with blood glucose levels in excess of 300 mg/dl were considered diabetic. The difference between the kinetics of disease in NOD and NOD IL-4 deficient mice was not statistically significant (p of log-rank test >0.05), irrespectively of treatment status. FIG. 17 shows the kinetics of disease in IL-4 deficient non-obese diabetic mice treated with insulin B formulated in spray dried particles and demonstrates a lack of dependency on IL-4 production in suppressing autoimmune diabetes in mice treated with Ins-SDLM IL4-ko.

### EXAMPLE 22

### Compositions Based on Spray Dried Particles Coformulated With Immune Modulatory Carbohydrates Such as Mannan

Preparation A was comprised of a perfluorocarbon-in-water emulsion of 6.2 g of perfluorooctyl bromide dispersed with the aid of 0.27 g of hydrogenated egg PC (EPC3, Lipoid) in 43 g of hot DI water with a T-25 Ultraturrax at 9000 rpm for about 5 min. The coarse emulsion was homogenized under high pressure (18,000 psi) for 5 discrete passes with an Avestin Emulsiflex C5. Preparation B contained 0.513 g of mannan purified from Saccharomyces Cerevisiae (Sigma Chemical Co). Preparation A was added to dissolve the ingredient in preparation B, now called preparation (A+B). Preparation C contained 50 mg of insulin B dissolved in 3.5 mL of water pH 3 (with the aid of HCl). Alternatively, the preparation contained 10 mg of UV-inactivated sucrose gradient-purified WSN influenza virus (A/WSN/32 H1N1 strain). Eight and a half grams of preparation A+B was added to preparation C. The combined feed preparation was spray dried with a standard B-191 Mini spray drier under the following conditions: inlet temperature=85° C.; outlet temperature=61° C.; aspirator=81%; pump=2.2 mL/min; and, nitrogen flow=2400 L/h. The theoretical final composition was: 25% EPC3; 50% mannan; and, 25% InsB; or, 45% EPC3; 50% mannan; and, 5% WSN antigen.

### EXAMPLE 23

### Increased Th2 Response to Antigen Coformulated With Mannan

BALB/c mice (2 month old females from Harlan; n=4/group) anesthetized with METOFANE were instilled with 40 μl of SDLM suspension containing 200 μg of mannan (made according to Ex. 22) and 20 μg of UV-killed WS virus. As controls, mice treated with HES-SDLM loaded with viral antigen (devoid of mannan) were used, instilled with dose-matched non-formulated killed (“sal-WSN”) or injected with live virus in the peritoneal cavity. At 14 days, the lungs and spleens were harvested and T cell profile assessed by ELISPOT analysis.

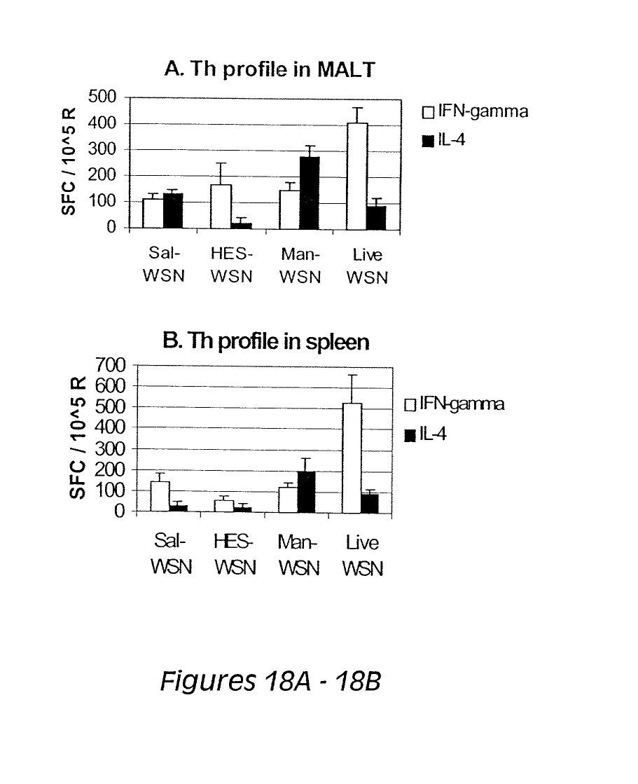

Lungs were harvested from treated mice, fragmented and digested using collagenase for 45 minutes at 37° C. in DMEM-1% BSA. The partially digested fragments were squeezed against 70 μm strainers (Falcon) and the liberated interstitial cells were collected in Petri dishes. The assay was carried out using nitrocellulose plates (Millipore) precoated with 4 μg/ml of anti-IFNγ or anti-IL-4 rat anti-mouse monoclonal antibodies (BD-PharMingen). Lung responder cells (A) or splenocytes (B) were stimulated in vitro with mitomycin-treated syngeneic APC in the presence of 5 μg/ml UV-WSN antigen or nil. Generally, the assay was carried out with using serial dilutions of cell suspension, ranging from 4×10⁵ to 5×10⁴ responder cells/well. The number of stimulator cells was 2×10⁵ cells/well. After 72 hours, the assay was developed using sequential steps of washing, incubation with 2 μg/ml of biotinylated anti-cytokine antibodies (BD-PharMingen), streptavidin-HRP and insoluble substrate (AEC). The frequency of spot-forming-colonies (SFC) was measured using an automatic acquisition system equipped with Image-Pro Plus software. In FIGS. 18A-18B, the results are expressed as number of cytokine⁺ SFC/10⁵ responder cells after subtraction of background against nil. FIGS. 18A-18B show increased IL-4 response (see Man-WSN in comparison to controls, e.g., “Sal-WSN” and “HES-WSN”) to virus antigen formulated into lipid microparticles together with mannan.

### EXAMPLE 24

### Enhanced Th2 Responses to Antigens Coupled to Carbohydrates that Bind to Lectin Receptors on Antigen Presenting Cells

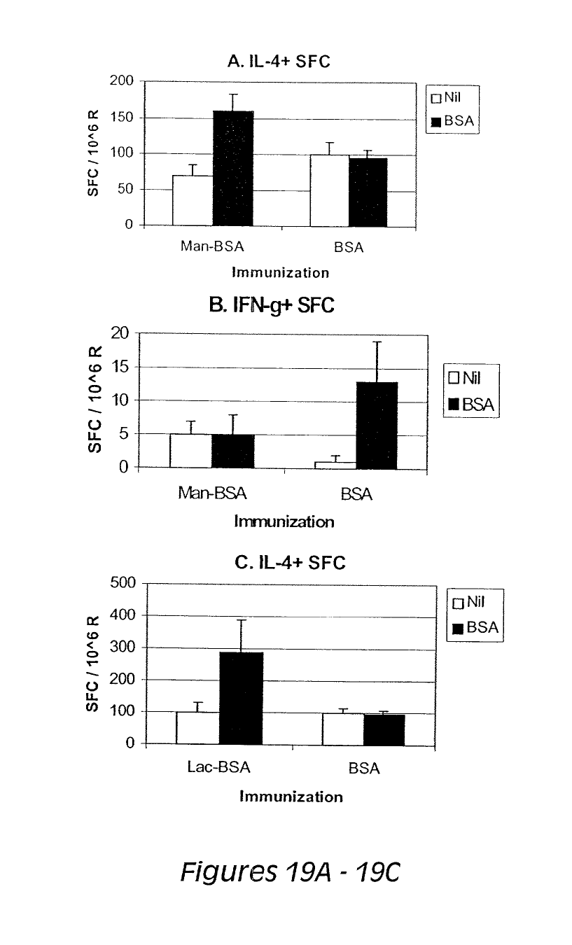

BALB/c mice were immunized intraperitoneally with 100 μg of BSA conjugated to mannose (FIGS. 19A-B) or lactose (FIG. 19C) residues (Sigma), dissolved in normal saline. The conjugates contained approximately 25 moles of mannose or lactose/mole of BSA. As a control, BSA not conjugated to carbohydrate residues were used. At 14 days, the mice were sacrificed and spleens removed. The T cell profile was assessed by ELISPOT analysis. The assay was carried out using nitrocellulose plates (Millipore) precoated with 4 μg/ml of anti-IFNγ or anti-IL-4 rat anti-mouse monoclonal antibodies (BD-PharMingen). Splenocytes were stimulated in vitro with mitomycin-treated syngeneic APC in the presence of 10 μg/ml of BSA antigen or nil. Generally, the assay was carried out using serial dilutions of cell suspension, ranging from 4×10⁵ to 5×10⁴ responder cells/well. The number of stimulator cells was 2×10⁵ cells/well. After 72 hours, the assay was developed using sequential steps of washing, incubation with 2 μg/ml of biotinylated anti-cytokine antibodies (BD-PharMingen), streptavidin-HRP and insoluble substrate (AEC). The frequency of spot-forming-colonies (SFC) was measured using an automatic acquisition system equipped with Image-Pro Plus software. The results are expressed in FIGS. 19A-19C as number of cytokine⁺ SFC/10⁶ responder cells. FIGS. 19A-19C show the induction of IL-4 producing T cells by carbohydrate conjugates of bovine serum albumin (“Man-BSA” and “Lac-BSA”).

### EXAMPLE 25

### Suppression of Autoimmune Disease by Administration of Self Antigen Coformulated With Mannan into Spray Dried Particles, and Delivered to Non-Obese Diabetic Mice

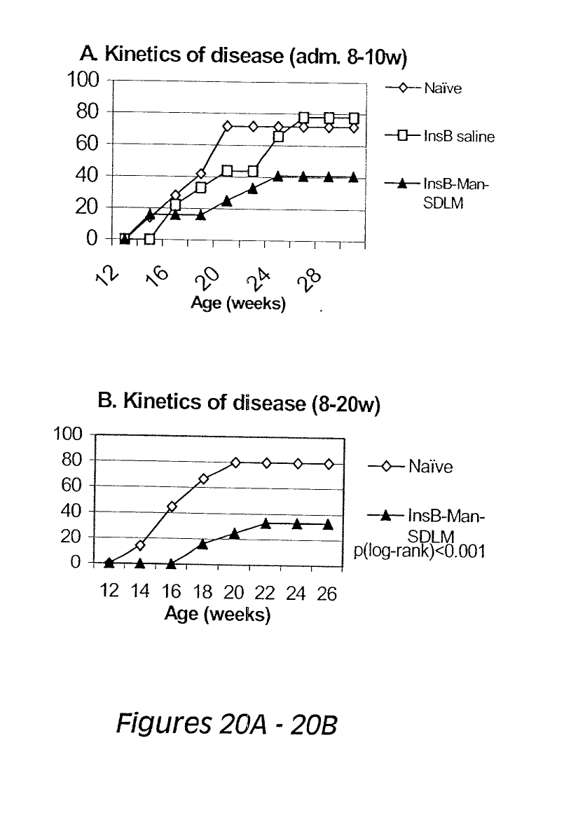

Female NOD mice (n=8/group) were anesthetized with METOFANE® and dosed via nostrils (weekly at the age of 8, 9, 10 weeks—FIG. 20A; or 8-20 weeks—FIG. 20B) with SDLM (400 μg/40 μl in perflubron-Liquivent®) containing 10% mannan and 25% of insulin B chain (InsB) (made according to Ex. 22) corresponding to 100 μg of antigen. In parallel, a group of mice was treated with dose-matched non-formulated InsB in saline. The kinetics of diabetes was determined by measuring blood glucose levels weekly, starting with the age of 12 weeks. Mice with blood glucose levels in excess of 300 mg/dl were considered diabetic. The mice treated with mannan-SDLM loaded with InsB were significantly protected from disease (FIG. 20A: p log-rank test <0.05 and FIG. 20B: p log-rank<0.001; compared to naive female NOD mice). No significant protective effect of non-formulated InsB was noted. The results of FIGS. 20A and 20B show suppression of autoimmune diabetes in non-obese diabetic mice treated with spray dried microparticles coformulated with mannan and insulin B chain.

### EXAMPLE 26

### Modification of Autoreactive T Cell Response by Treatment With Lipid Microparticles Loaded With Insulin B Chain and Mannan

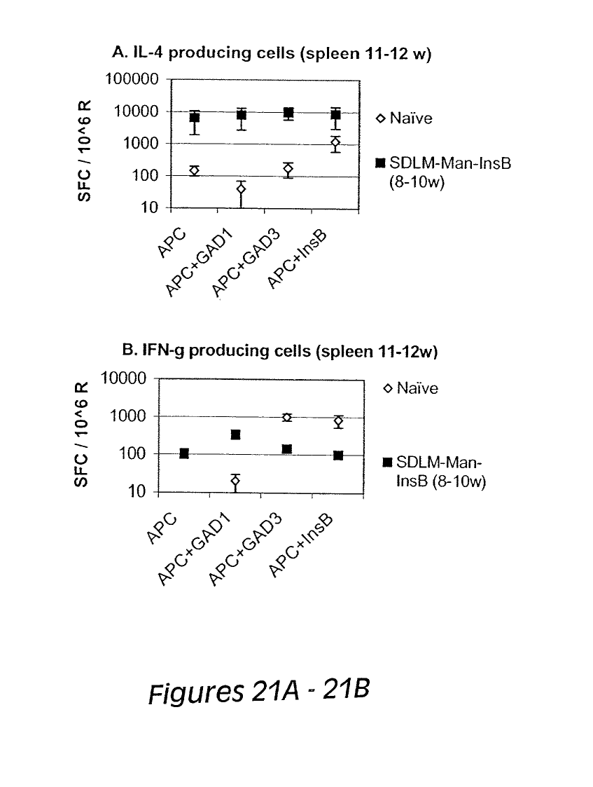

Mice were treated at the age of 8, 9 and 10 weeks with 25%-InsB-SDLM co-formulated with 10% mannan (made according to Ex. 22), delivered by nasal instillation, at a dose of 400 μg of formulation in 40 μl of PFOB. As controls, naive female NOD mice were used. Spleens were harvested at the age of 12-13 weeks. Single cell suspension was prepared from each spleen (n=4/group) and after hypotonic lysis of red blood cells, the splenocytes were resuspended in HL-1 medium. The splenocytes were incubated for 5 days with InsB or GAD-65-derived peptides, GAD1 or GAD3 from Research Genetics (5×10⁶ cells/3 ml, in the presence of 20 μg/ml of peptide). After the preliminary incubation with peptides, the T cells were expanded by replacing the cell culture medium with fresh HL-1 medium supplemented with 20U/ml of rIL-2. Following standard cell culture incubation for 3 days, the cells were transferred to ELISPOT plates precoated with 4 μg/ml anti-cytokine antibodies (PharMingen). The stimulation was carried out in the presence of mitomycin treated nod splenocytes as feeder cells (2×10⁵ cells/well) as well as InsB or GAD-65-derived peptides (20 μg/ml). After 72 hour incubation, the ELISPOT assay was developed using biotinylated anti-cytokine antibodies, followed by addition of streptavidin/horse-radish peroxidase conjugate and insoluble substrate (AEC). The data was acquired and processed using a CDC camera connected to a computer operating ImagePro 4.1 image analysis software. The results are expressed in FIGS. 21A-21B as number of cytokine producing cells/10⁶ responder cells and show the modification of autoreactive T cell immunity by administration of insulin B coformulated with mannan in SDLM in non-obese diabetic mice.

### EXAMPLE 27

### Compositions Based on Spray Dried Particles Formulated With Immune Modulatory Carbohydrates Such as Mannan and Devoid of Protein Antigens

Preparation A was comprised of a perfluorocarbon-in-water emulsion of 6.2 g of perfluoroctyl bromide dispersed with the aid of 0.513 g of hydrogenated egg PC (EPC3, Lipoid) in 43 g of hot DI water with a T-25 Ultraturrax at 9000 rpm for about 5 min. The coarse emulsion were homogenized under high pressure (18,000 psi) for 5 discrete passes with an Avestin Emulsiflex C5. Preparation B contained 0.513 g of mannan (Sigma Chemical Co). Preparation A was added to dissolve the ingredient in preparation B, now called preparation (A+B). Preparation C contained 3.5 mL of water. Eight and a half grams of preparation A+B was added to preparation C. The combined feed preparation was spray dried with a standard B-191 Mini spray drier under the following conditions: inlet temperature=85° C.; outlet temperature=61° C.; aspirator=81%; pump=2.2 mL/min; and, nitrogen flow=2400 L/h. The theoretical final composition was: 50% EPC3 and 50% mannan.

### EXAMPLE 28

### Beneficial Effect of Spray Dried Microparticles Loaded With Mannan and Devoid of Protein Antigens

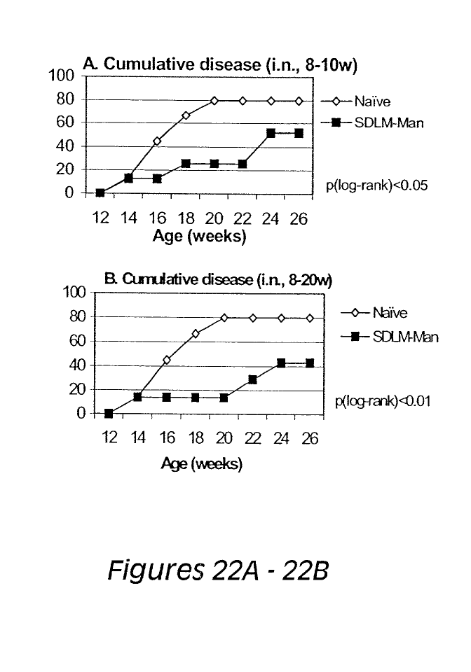

Female NOD mice (n=8/group) were anesthetized with METOFANE® and dosed via nostrils (weekly at the age of 8, 9, 10 weeks—FIG. 22A; or 8-20 weeks—FIG. 22B) with SDLM (400 μg/40μl in perflubron-Liquivent®) containing 50% mannan (made according to Ex. 22). The kinetics of diabetes was determined by measuring blood glucose levels weekly, starting with the age of 12 weeks. Mice with blood glucose levels in excess of 300 mg/dl were considered diabetic. The mice treated with mannan-SDLM were significantly protected from disease (p log-rank test <0.05; compared to naive female NOD mice). The results in FIGS. 22A-22B show the protective effect of SDLM with mannan and devoid of self-antigen on disease in non-obese diabetic mice.

### EXAMPLE 29

### Effect of Microparticles Loaded With Mannan and Devoid of Self Antigen on the Profile of T Cells from Non-Obese Diabetic Animals

Female NOD mice were treated by nasal instillation at 8, 9 and 10 weeks with SDLM containing 10% mannan or 10% mannan and 25% insulin B chain, as detailed above in examples 27 and 22, respectively. The animals were sacrificed at 12-13 weeks and non-diabetic ones at 30-31 weeks, respectively. As controls, naïve female NOD mice were used.

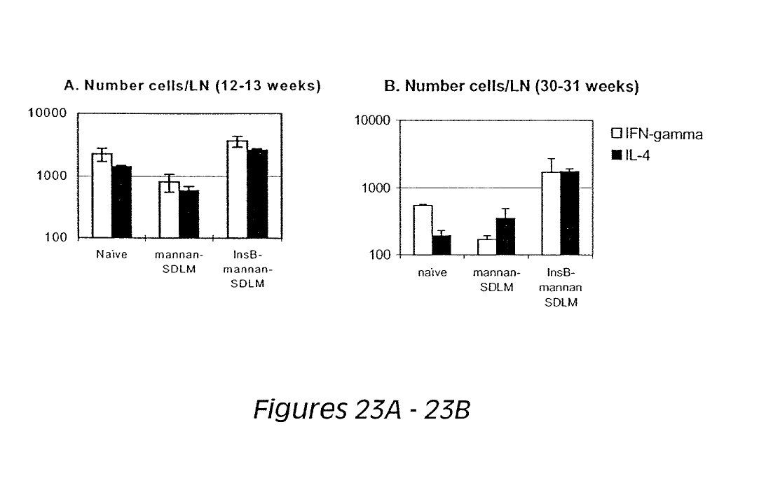

Peri-pancreatic lymph nodes were harvested and placed immediately in ice-cold DMEM medium. The lymph nodes were pooled from 3-4 animals/group. Subsequently, they were incubated in DMEM-0.45% BSA supplemented with collagenase, for 1 hour at 37° C. A single cell suspension was obtained by passing through Falcon strainers and the cells were incubated in ELISPOT plates previously coated with anti-IFN-γ or anti-IL-4 antibodies (PharMingen), at concentrations of 2, 1, 0.5 and 0.25×10⁵ cells/100 μl in HL-1 medium and in the presence of 5 μg/ml of anti-CD3 mAb+2 μg/ml of anti-CD28 mAb (PharMingen). After 48 hours of incubation under standard cell culture conditions, the cells were washed off and the assay was read using biotinylated anti-cytokine antibodies and subsequently, streptavidin-horseradish peroxidase (BioSource) and insoluble substrate (AEC, Sigma). The experiment was carried out in triplicates. The results are expressed in FIGS. 23A-23B as number of SFC (means ±SEM) normalized to the average number of pancreas lymph node cells/mouse (approximately 3.1×10⁶). The results in FIGS. 23A-23B show the Th1 to Th2 shift of cell profile in lymph nodes from treated mice and the effect of mannan-SDLM on the profile of autoreactive T cells from NOD mice. Particularly significant is the increase in IL-4 production in mice treated with mannan-SDLM over the naive (control) as shown in FIG. 23B.

### EXAMPLE 30

### Compositions that Limit the Immune Response Against Formulated Macromolecules

The process of making the compositions was described in previous examples 1, 14, 16, 22 and 27. Preferred compositions associated with limited immunity are obtained with spray drying temperatures of less than 100° C. (preferably less than 60° C.). The compositions may be 1-80% surfactant by weight (e.g. surfactants can be chosen from phosphatides, non-ionic surfactants, cationic surfactants, anionic surfactants and proteins, amino acids and oligoaminoacids that are known to exhibit surfactant properties); 10-50% excipient by weight (excipients may be selected from carbohydrates, salts, proteins and/or synthetic polymers); 0-80% bioactive substance by weight and a molar ratio of metal ion to phosphatides of 0-2.

Preferred surfactants include phosphatides: homo and heterochain phosphatidylcholines (PC's), phosphatidylserines (PS's), phosphatidylethanolamines (PE's), phosphatidylglycerols (PG's), phosphatidylinositols (PI's), sphingomyelins, gangliosides, 3-Trimethylammonium-Propane phosphatides (TAP's) and Dimethylammonium-Propane phosphatides (DAP's), having hydrocarbon chain length ranging from 5 to 22 carbon atoms. One (lysophosphatides) or double chain surfactants are also contemplated. The phosphatides may be hydrogenated, unsaturated or partially hydrogenated. The most preferred phosphatides are natural phosphatides derived from soy or egg, hydrogenated phosphatides derived from soy and egg, DiC18PC, DiC16PC, DiC14PC, DiC8PC DiC6PC, DiC16PS DiC14PS, DiC8PS and DiC6PS.

Contemplated non-ionic surfactants include poloxamers, tweens, tritons, PEG's, sugar esters. Most preferable non-ionic surfactants are poloxamer 188, poloxamer 407, tween 80, PEG 1540 cetyl alcohol and tyloxapol. Cationic surfactants may include benzalkonium chloride. Anionic surfactants may be selected from the cholate and deoxycholate family, like CHAPS, taurocholate, deoxytaurocholate, or phosphate fatty acid salts such as dicetyl phosphate. Other surface active compounds include albumin, Leucine, oligopeptides, oligoleucine, oligoalanine, saponins, (for a better listing see Gower's handbook of industrial surfactants 1993, pages 885-904, ISBN 0566074575), etc.

Preferred excipients for the compositions include carbohydrates such as, lactose, mannitol, mannose, galactitol, raffinose, maltose, glucose, saponins, sorbitol, sucrose, trehalose, cellulose and derivatives, pectins, dextrans, chitosan, chitin, mucopolysaccharides, chondroitin sulfate, saponins etc. Proteins include albumin (human, egg or bovine). Synthetic polymers like PEGs and poloxamers, etc. Other excipients include osmotic agents such as NaCl, KCl, magnesium chloride, calcium chloride, etc. and buffer systems like PBS, acetate, tris, etc.

Preferred metal ions include metal ions or salts from groups IIa, IIIa and metal ions from atomic numbers 21-30; 39-48, 57-80 and 89-106. The preferred metal ions are calcium, magnesium, aluminum and zinc.

### EXAMPLE 31

### Limiting the Immune Response of Immunoglobulins by Modifying the Microparticle Composition

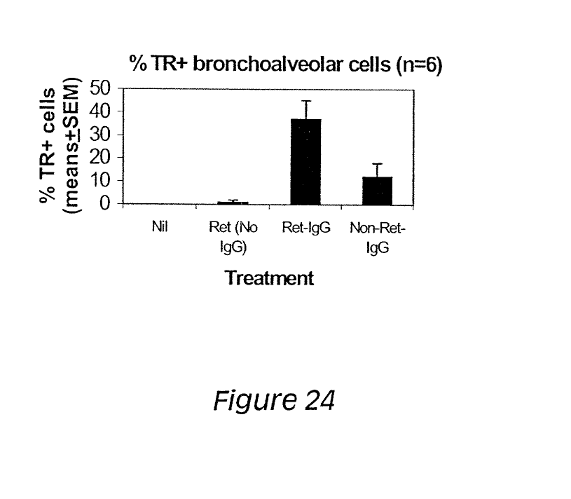

Retentive (10% lactose, 25% hIgG and 64% DPPC+1% DiC16PE-Texas Red) and non-retentive particles (1% tyloxapol, 10% lactose, 25% hIgG and 63% DPPC⁺Ca²⁺+1% DiC16PE-Texas Red) (SDLMs constructed according to the teachings of Example 1, using excipients mentioned in this example) were administered to anesthetized Sprague-Dawley rats, using an Insufflator device (Penn Century) inserted into the trachea. Prior to administration, the device was loaded with 60 μl of 20 mg/ml formulation suspended in perflubron (PFOB, Liquivent®). Out of 60 μl, 35 μl were expelled by the device as micron-size aerosols. One hour after administration, the rats (n=6/group) were sacrificed and the bronchoalveolar macrophages harvested, washed and analyzed using a FACS Calibur (Becton Dickinson). The percentage of Texas Red positive cells was measured using cells from naive rats as reference. The results are expressed as % Texas Red⁺ cells (means ±SEM)(see FIG. 24).

In parallel, the lung circulation was perfused via the right ventricle. The lungs were harvested, homogenized in 10 μg/ml of aprotinin (10 ml final volume) and the concentration of hIgG measured by capture ELISA. Briefly, wells coated with 1:500 mouse anti-human k and λ chains antibodies (Sigma Immunochemicals) were used, blocked subsequently with 30% SeaBlock (Pierce). After the centrifugation of tissue homogenates (5 minutes at 10,000 RPM), various dilutions of supernatants were incubated for 2 hours at room temperature. Following extensive washing, the wells were incubated for 1 hour at room temperature with 1:1000 goat anti-human IgG conjugated with alkaline phosphatase (Sigma Immunochemicals). Subsequently, the assay was developed using pNPP substrate according to manufacturer's instructions (Sigma). The OD was read at 405 nm and the concentration of hIgG calculated by interpolation using a standard curve constructed with hIgG (Sigma).

The results are expressed in FIG. 24 as total amount of IgG in the lung interstitial tissue (means ±SEM). The results show that co-formulation of various excipients is associated with differential clearance of microparticles by phagocytes. Since this clearance is FcγR-mediated, the results demonstrate that co-aggregation of IgG with lipid excipients is greatly limited by addition of tyloxapol and modification of excipients.

### EXAMPLE 32

### Limiting the Antibody Response to Formulated Immunoglobulins

BALB/c mice were immunized by intranasal administration of microparticles resuspended in perfluorocarbon (PFOB, Liquivent®) at a dose of 800 μg in 40 μl. Two different formulations were administered: retentive particles (containing 10% lactose, 25% hIgG and 65% DPPC with use of perfluorocarbon during manufacturing); and non-retentive particles composed of 1% tyloxapol, 10% lactose, 25% hIgG and 64% of DPPC without the use of perfluorocarbon during the technological process). At 7 and 21 days after administration, blood was harvested, serum separated and the titer of anti-hIgG antibodies measured by ELISA assay. In brief, hIgG was coated at 10 μg/ml onto plastic wells, with subsequent blocking using 30% SeaBlock (Pierce). Various serum dilutions were incubated in wells for 2 hours at room temperature and after extensive washing, 1:1000 goat anti-mouse IgG antibodies coupled with alkaline phosphatase (Sigma Immunochemicals) were incubated for 1 hour at room temperature. The reaction was developed with pNPP and read at OD 405 nm using an automatic ELISA reader.

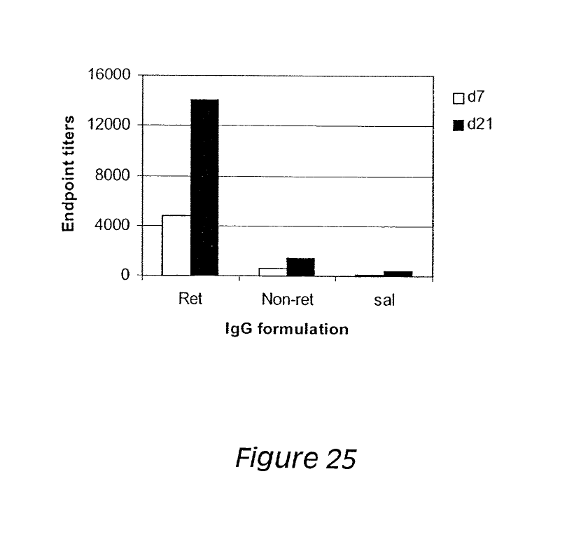

The results are expressed as means of endpoint titers (n=4/group) in FIG. 25. The results show that microparticles containing similar amounts of IgG resulted in different levels of antibody production. Less retentive particles resulted in more limited immune responses, in concordance with a less interaction of the proteins with the excipients.

### EXAMPLE 33

### Limiting the Th Response to Formulated Immunoglobulin

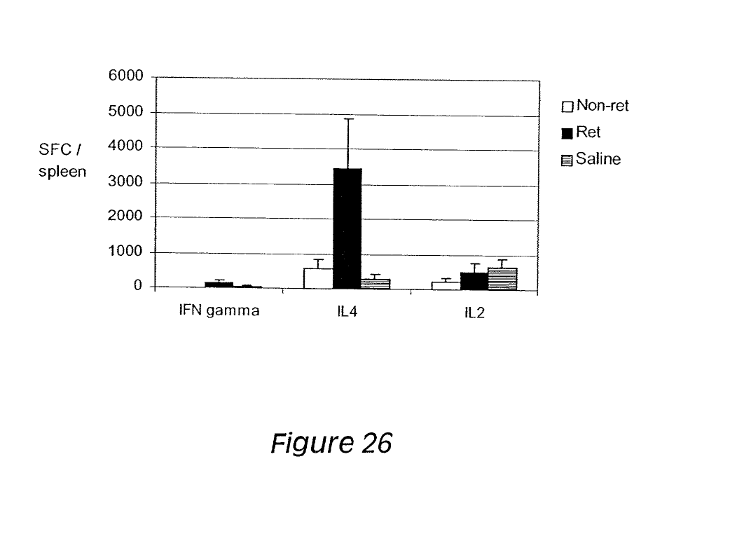

BALB/c mice were immunized by intranasal administration of microparticles resuspended in perfluorocarbon (PFOB, Liquivent®) at a dose of 800 μg in 40 μl. Two different formulations were administered: retentive particles composed of 10% lactose, 25% hIgG and 65% DPPC with use of perfluorocarbon during technological process and non-retentive particles composed of 1% tyloxapol, 10% lactose, 25% hIgG and 64% of DPPC-Ca²⁺ without the use of perfluorocarbon during the technological process. The administration of microparticles was repeated at day 7 and 14 and the mice were sacrificed on day 21. Splenocytes were isolated and incubated (5×10⁵ cells/well) with human IgG (20 μg/ml) in nitrocellulose wells precoated with 4 μg/ml of anti-IFN-γ, anti-IL-4 or anti-IL-2 rat anti-mouse monoclonal antibodies (PharMingen BD). After 72 hours of incubation at 37° C. and 5% CO₂, the cells were washed off and the wells were incubated overnight at 4° C. with 2 μg/ml of biotinylated anti-cytokine antibodies (PharMingen BD). Subsequently, the reaction was developed with 1:1000 streptavidin-HRP (BioSource) and AEC substrate (Sigma). The frequency of spot forming colonies (SFC) was automatically measured using ImagePro system and the final results are expressed in FIG. 26 as total number of cytokine-SFC/spleen (means ±SEM of triplicates). The results show different formulations of same protein (hIgG) is associated with different T cell response. Less retentive particles result in more limited induction of Th2 responses.

### EXAMPLE 34

### Preparation of Protein Formulations Using DPPC as the Main Surfactant and Modifying the Particle Nature by the Addition of High HLB Surfactants

The following compositions (or similar compositions to the ones described herein) are contemplated to be administered to the respiratory tract by liquid dose instillation, nebulization, aerosolization, dry powder inhalation and metered dose inhalation as well as the reconstituted composition in water (or any suitable solvent or mixture of solvents that will dissolve or suspend the composition), as well as in an non-aqueous media.

Sample HL

Preparation A was comprised of a liposome suspension of 0.48 g of DPPC dispersed in 23 g of hot DI water with a T-25 Ultraturrax at 9000 rpm for about 5 min. The coarse liposomes were homogenized under high pressure (18,000 psi) for 5 discrete passes with an Avestin Emulsiflex C5. Preparation B contained 0.12 g of CaCl₂.2H₂O, 0.012 g of tyloxapol and no lactose monohydrate. Preparation A was added to dissolve all the ingredients in preparation B, now called preparation (A+B). Preparation C contained 595 mg of bovine IgG (Calbiochem, San Diego Calif.) dissolved in 6 mL of 0.9% NaCl.

Preparation A+B (after it cooled down to room temperature) was added to preparation C. The combined feed preparation was spray dried with a standard B-191 Mini spray drier equipped with a modified high efficiency cyclone under the following conditions: inlet temperature=70° C.; outlet temperature=44° C.; aspirator=89%; pump=2.2 mL/min; and nitrogen flow=2400 L/h. The theoretical final composition was: 40% DPPC; 50% Bovine IgG; 10% calcium chloride dihydrate; and, 1% tyloxapol.

The following preparations were also made with the same process described above:

| | | | | | | |
|---|---|---|---|---|---|---|
| | | | | CaCl₂. | | Bovine |
| Sample | DPPC | DiC8PC | Tyloxapol | 2H₂O | Lactose | IgG |

Grams of material used

| | | | | | | |
|---|---|---|---|---|---|---|
| HL | 0.48 | 0.000 | 0.012 | 0.120 | 0.000 | 0.595 |
| BLAC | 0.14 | 0.000 | 0.012 | 0.036 | 0.405 | 0.593 |
| HLNT | 0.48 | 0.000 | 0.000 | 0.120 | 0.000 | 0.592 |
| P8LT | 0.12 | 0.025 | 0.000 | 0.036 | 0.417 | 0.595 |

% theoretical composition

| | | | | | | |
|---|---|---|---|---|---|---|
| HL | 40 | 0 | 1 | 10 | 0 | 50 |
| BLAC | 12 | 0 | 1 | 3 | 34 | 50 |
| HLNT | 40 | 0 | 0 | 10 | 0 | 50 |
| P8LT | 10 | 2 | 0 | 3 | 35 | 50 |

Particle size measurement was performed on the BLAC composition by dry powder inhalation using a commercial DPI device called the FlowCaps (Hovione, Portugal). Approximately 12 mg of powder was loaded in 2 capsules and the powder was delivered to an eight stage Andersen Cascade Impactor with an induction port. The particle size measurement was performed at an inspiration flow rate of 28.3 L/min. for a period of 5 seconds. Each stage was extracted with a saline-SDS solution (0.45%, 0.25%) in water. The amount of protein in each stage was quantified using the Bio-Rad DC Protein assay kit, against a calibration curve performed using fresh bovine IgG (Calbiochem, San Diego, Calif.) that was dissolved in 0.45% saline and 0.25% SDS. The percent emittance from the capsule was above 90% and the fine particle fraction (FPF) was of 65% and the mean mass aerodynamic diameter (MMAD) of 3.74μm with a geometric standard deviation (GSD) of 2.19 μm.

A similar composition to BLAC, but this time having a protein concentration of 68%, was formulated as an MDI suspension in HFA 134a. The suspension resembled milk and no creaming or sedimenting was observed within 5 minutes. Particle size distribution was performed with 50 shots to an 8 stage ACI, and the amount of protein in each stage was quantified as outlined above. The MMAD was of 3.7 μm with a GSD of 1.83 μm and a FPF of 63%.

### EXAMPLE 35

### Shows the Release of Bovine IgG from Formulations that Could Induce an Immune Response and Formulations that Could Not

This Example is utilizing bovine IgG, but any other bioactive substance, such as peptides, proteins, nucleic acids, nucleotides, etc. are contemplated to behave in a similar matter. The protein release rate from the spray dried powders from example (34) were analyzed as follows:

Ten milligrams of each powder was delivered to a 2 mL Eppendorf micro-centrifuge tube, and the powder was forced down by centrifuging the powder for ten minutes at 10,000 rpm in a microcentrifuge. To each sample, one mL of a 0.9% saline solution was added and a 100 μl sample was taken after rocking the sample in a “vari-mix from Thermolyne” at its maximum speed of 20 cycles/min. at the following time points: 0.5, 1, 2, 5, 10, 15, 30, 60 minutes or until dissolved. The samples that took more than 60 minutes to dissolve, one mL of a solution containing 0.45% saline and 0.25% SDS was added to the microcentrifuge tube in order to fully dissolve the composition.

One hundred microliters of fresh 0.9% saline was added to the micro-centrifuge tube after each sampling. Before quantification, 100 μl of a solution containing 0.45% saline and 0.25% SDS was added to each sample. The protein quantification was performed using the Bio-Rad DC Protein assay kit, against a calibration curve performed using fresh bovine IgG (Calbiochem, San Diego, Calif.) that was dissolved in 0.45% saline and 0.25% SDS.

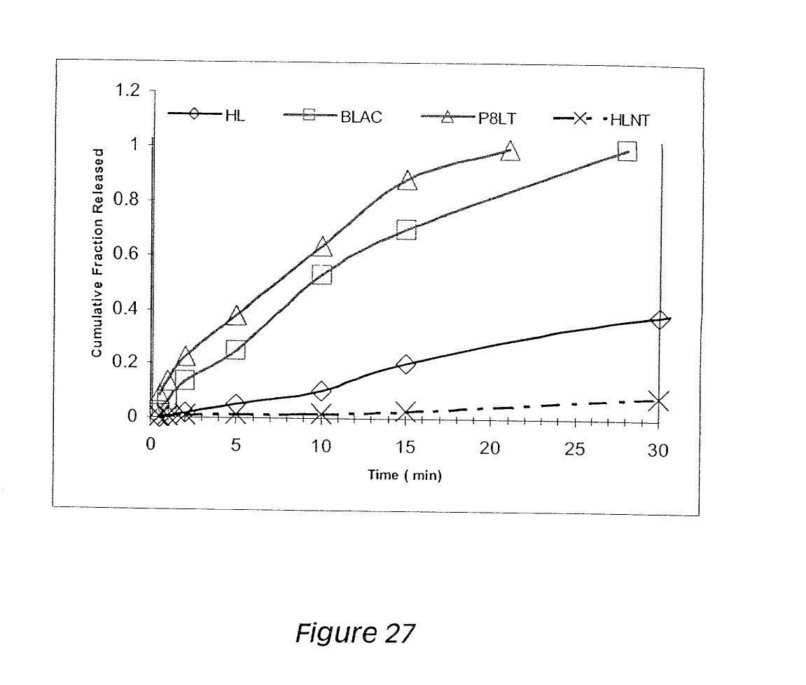

The results in FIG. 27 show two distinctive formulations, one called retentive particles (HL and HLNT) that can upregulate, redirect, etc the immune response, and another called non-retentive particles (BLAC and P8LT) that can limit the immune response to the bioactive delivered.

### EXAMPLE 36

### Effect of Excipients on the Release Rate of Bovine IgG Compositions

The following compositions were tested for the release rate of bovine IgG:

1) Pure bovine IgG (Calbiochem, San Diego Calif.); and,

2) Bovine IgG saline powder 300 mg of bovine IgG was dissolved in 3 mL of a 0.9% NaCl solution, then follow by evaporation to dryness under vacuum.

Ten milligrams of each powder was delivered to a 2 mL Eppendorf micro-centrifuge tube, and the powder was forced down by centrifuging the powder for ten minutes at 10,000 rpm in a microcentrifuge. To each sample, one mL of a 0.9% saline solution was added and a 100 μl sample was taken after rocking the sample in a “vari-mix from Thermolyne” at its maximum speed of 20 cycles/min. at the following time points: 0.5, 1, 2, 5, 10, 15, 30, 60 minutes or until dissolved. The samples that took more than 60 minutes to dissolve were processed by adding one mL of a solution containing 0.45% saline and 0.25% SDS to the microcentrifuge tube in order to fully dissolve the composition.

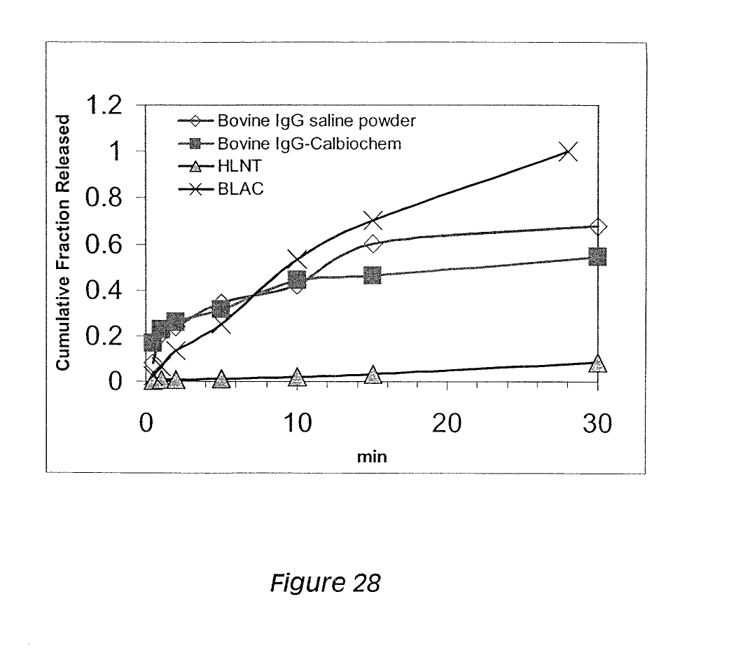

One hundred microliters of fresh 0.9% saline was added to the micro-centrifuge tube after each sampling. Before quantification, 100 μl of a solution containing 0.45% saline and 0.25% SDS was added to each sample. The protein quantification was performed using the Bio-Rad DC Protein assay kit, against a calibration curve performed using fresh bovine IgG (Calbiochem, San Diego, Calif.) that was dissolved in 0.45% saline and 0.25% SDS. BLAC and HLNT compositions from Example 34 were added to FIG. 28 for comparison only.

The results show how by increasing the amount of water soluble excipients as well as surfactants, the release of the bioactive substance from powdered compositions can be modified. This can be useful in controlling (upregulate, redirect or limit) immune responses to nucleic acids, nucleotides, peptides and proteins.

### EXAMPLE 37

### Measurement of the “Particle” Characteristics of Different Bovine IgG Formulations After Fully Hydration in Water

Example 37 utilizes bovine IgG, but any other bioactive compound, such as peptides, proteins, nucleic acids, nucleotides, etc. are contemplated to behave in a similar matter.

A. Preparation of Sample C8PC.

Preparation A was comprised of a liposome/micellar suspension of 0.14 g of DiC8PC dispersed in 23 g of hot DI water. Then 0.04 g of CaCl₂.2H₂O and 0.405 g of lactose monohydrate was added until dissolved, the resulting preparation was a clear solution. Preparation B contained 595 mg of bovine IgG (Calbiochem, San Diego Calif.) dissolved in 6 mL of 0.9% NaCl. Preparation A (after it cooled down to room temperature) was then added to preparation B. The combined feed preparation was spray dried with a standard B-191 Mini spray drier equipped with a modified high efficiency cyclone under the following conditions: inlet temperature=70° C.; outlet temperature=44° C.; aspirator=89%; pump=2.2 mL/min.; and, nitrogen flow=2400 L/h. The theoretical final composition was: 12% DiC8PC; 50% Bovine IgG; 3% calcium chloride dihydrate; and 34% lactose monohydrate.

Approximately 10 mg of each of the samples were weighted out in a 5 mL test tube. 2 mL of a saline solution (0.9% NaCl) was added to each sample, and the sample was mixed for at least 60 minutes before analysis. The “particle” characteristic of each composition was measured by visible spectrophotometry. The measurement is based on “particles” (defined here as of colloidal origin, that is, solid in gas; liquid in liquid; liquid in gas; solid in liquid; etc that are capable of scattering light). Colloidal particles that are able to scatter visible light, are particles that are typically in the submicron size range (i.e., 0.1 μm and above). These “particles” can interact via hydrophobic interactions with the bioactive components in the formulation and control (upregulate, redirect) the immune responses to nucleic acids, nucleotides, peptides and proteins.

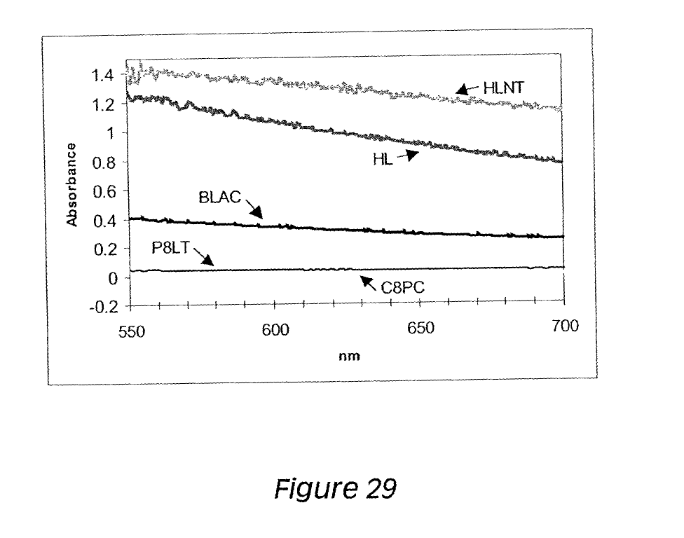

The results in FIG. 29 show that the compositions with larger amounts of a non-water soluble surfactant (or mixture of surfactants, having and overall low HLB) with small or no water soluble ingredients that have a high water diffusion rate will tend to form retentive particles, while the compositions that are formulated with surfactants with a higher HLB (higher water solubility and higher diffusion in water) and with a larger amount of water soluble ingredients (having a high diffusion rate in water) will tend to form non-retentive particles.

### EXAMPLE 38

### Preparation of Protein Compositions Varying the Surfactant and the Concentration of the Surfactant

The following compositions (or similar compositions to the ones described herein) are contemplated to be administered to the respiratory tract by liquid dose instillation, nebulization, aerosolization, dry powder inhalation and metered dose inhalation of the reconstituted composition in water (or any suitable solvent or mixture of solvents that will dissolve or suspend the composition), as well as the powder itself, or in an non-aqueous media. These compositions are designed to control (upregulate, redirect or limit) immune responses to nucleic acids, nucleotides, peptides and proteins.

A. Sample 1Tch

Preparation A: 286 mg of bovine IgG was dissolved in 3 mL of Dulbecco's PBS buffer. Preparation B: 9.5 mg of taurocholate Na dihydrate and 0.657 gr of Lactose monohydrate was dissolved in 19 mL of DI water. Preparation A was mixed with preparation B and mixed thoroughly. An aliquot of about one mL was withdrawn and transferred to a 10 mL scintillation jar and frozen before lyophilization. The rest of the combined preparation was spray-dried with a standard B-191 Mini spray drier equipped with a modified high efficiency cyclone under the following conditions: inlet temperature=70° C.; outlet temperature=44° C.; aspirator=89%; pump=2.2 mL/min. and, nitrogen flow=2400 L/h.

The following preparations were also made with the same process described above. Approximate amount of material used (grams):

| Sample | Surfactant^{a} | | Bovine IgG | Lactose | Polymer^{b} | |
|---|---|---|---|---|---|---|
| 1Tch | 0.0095 | (Tch) | 0.286 | 0.657 | — | |
| 10Tch | 0.0956 | (Tch) | 0.290 | 0.571 | — | |
| 1Pol | 0.0095 | (Pol) | 0.286 | 0.657 | — | |
| 10Pol | 0.0956 | (Pol) | 0.290 | 0.571 | — | |
| 1DB | 0.0095 | (DB) | 0.286 | 0.657 | — | |
| 10DB | 0.0956 | (DB) | 0.290 | 0.571 | — | |
| Car | 0.0010 | (Pol)^{c} | 0.289 | 0.554 | 0.109 | (Car) |
| PVP | 0.0011 | (Pol) | 0.280 | 0.132 | 0.536 | (PVP) |
| CMC | 0.0011 | (Pol) | 0.280 | 0.550 | 0.106 | (CMC) |

^{a}Surfactants used: Tch = taurocholate Na dihydrate; Pol = poloxamer 188; DB = didodecyldimethylamonium bromide
^{b}Polymers used: Car = Carbopol 934P; PVP = polyvinylpyrrolidone 40K; CMC = Carboxymethyl cellulose Na salt, high viscosity
^{c}pH of the preparation containing the polymer was adjusted with 1 N NaOH to 6.7 before mixing with preparation A

Similar release rates were obtained with the lyophilized compositions. A tendency of a slight decrease in the dissolution time was observed, probably cause by the morphology of the composition. When the compositions/microparticles have irregularities that will increase the surface area and the surface that will be exposed to water or moisture, an increase in the release rate is expected.

The results show that by altering some of the excipients it is possible to create retentive particle (e.g., Car, CMC, etc) and non-retentive particles (e.g., 1Pol, 10Pol, 1Tch, etc.). Retentive particles are designed to control (upregulate, redirect) immune responses to the bioactive, while non-retentive compositions are designed to limit an immune response to the formulated bioactive compound. Examples of bioactives that need to be formulated in non-retentive formulation would be proteins or peptides for hormone replacement, such as insulin, growth hormones, calcitonin, etc.

### EXAMPLE 39

### Preparation of Insulin Compositions Varying the Surfactant and the Concentration of the Surfactant

A. Sample DINS

Preparation A was comprised of a liposome suspension of 0.02 g of DPPC dispersed in 10 g of hot DI water with a T-25 Ultraturrax at 9000 rpm for about 5 min. The coarse liposomes were homogenized under high pressure (18,000 psi) for 5 discrete passes with an Avestin Emulsiflex C5. Preparation B contained 0.005 g of CaCl₂.2H₂O, 0.005 g of tyloxapol and 0.30 g of lactose monohydrate. Preparation A was added to dissolve all the ingredients in preparation B, now called preparation (A+B).

Preparation C contained 0.143 g of insulin and was dissolved with the aid of 30% acetic acid. Preparation A and B was mixed, preparation A+B (after it cooled down to room temperature) was added to preparation C, then the pH adjusted with 2.2 mL of IN NaOH to a pH of 4.2. One mL of the combined feed preparation was frozen and lyophilized, the rest was spray-dried with a standard B-191 Mini spray drier equipped with a modified high efficiency cyclone under the following conditions: inlet temperature=70° C.; outlet temperature=47° C.; aspirator=89%; pump=2.2 mL/min. and, nitrogen flow=2400 L/h.

A. Sample 18DINS

Preparation A was comprised of a liposome suspension of 0.06 g of DPPC dispersed in 10 g of hot DI water with a T-25 Ultraturrax at 9000 rpm for about 5 min. The coarse liposomes were homogenized under high pressure (18,000 psi) for 5 discrete passes with an Avestin Emulsiflex C5. Preparation B contained 0.005 g of CaCl₂.2H₂O, 0.005 g of tyloxapol and 0.26 g of lactose monohydrate. Preparation A was added to dissolve all the ingredients in preparation B, now called preparation (A+B).

Preparation C contained 0.143 g of insulin and was dissolved with the aid of 30% acetic acid. Preparation A and B was mixed, preparation A+B (after it cooled down to room temperature) was added to preparation C, then the pH adjusted with 2.2 mL of IN NaOH to a pH of 4.2. One mL of the combined feed preparation was frozen and lyophilized, the rest was spray-dried with a standard B-191 Mini spray drier equipped with a modified high efficiency cyclone under the following conditions: inlet temperature=80° C.; outlet temperature=48° C.; aspirator=89%; pump=2.2 mL/min. and, nitrogen flow=2400 L/h.

B. Sample PINS

Preparation A was comprised of 0.0.164 g of lactose monohydrate, and 0.003 g of tyloxapol dissolved in 5 g of water. Preparation B was comprised of 0.143 g of insulin dissolved in 30% acetic acid. Preparation A was mixed with preparation B and 1.2 mL of a 1 N NaOH solution added to adjust the pH to 4.2. The combined feed preparation was frozen and lyophilized.

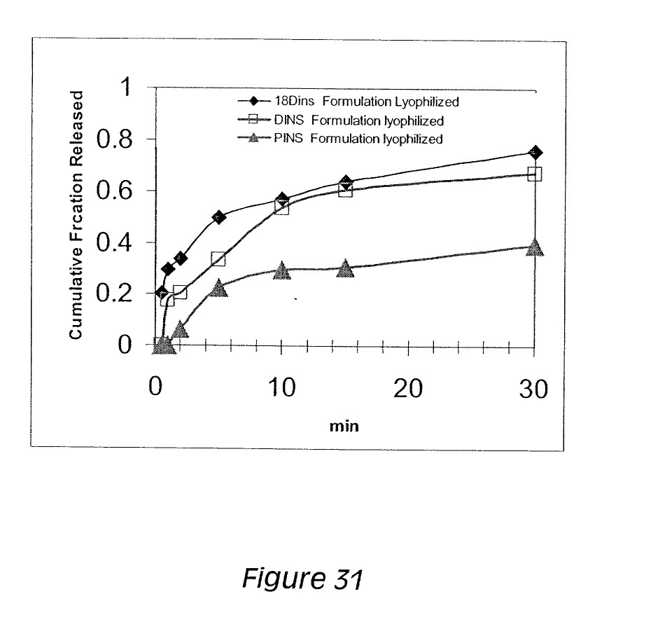

Less than 1 mg of the spray-dried powders was loaded to and active DPI (Penn century insufflator), and the particle size distribution for spray-dried samples 18DINS and DINS was measured by aerosizer DSP (TSI corporation) using the aerosampler attachment. The volume mean aerodynamic diameter was 2.98 μm ±1.49 for sample DINS and 2.49 μm ±1.49 for sample 18DINS. The release rate of insulin from the lyophilized compositions are shown in FIG. 31, the method employed is the same described in example 35.

The results show that and increase in insulin release is observed by increasing the amount of surfactant from 1% tyloxapol to 6% surfactant (5% DPPC and 1% Tyloxapol) or more. Increasing the concentration of surfactant to 13% (12% DPPC and 1% tyloxapol) did not greatly increase the release rate of insulin. This is probably caused by the low solubility of insulin at neutral pH.

What is claimed is:

1. A microparticle composition for controlling an immune response by downregulating a pathothegic arm of the immune system, or upregulating the suppressor arm of the immune system, or simultaneously downregulating the pathogenic arm and upregulating the suppressor arm of the immune system comprising:
a surfactant or mixture of surfactants comprising approximately 1-80% of the weight of the total microparticle composition;
at least one excipient selected from the group consisting of carbohydrates, polyols, salts, proteins and synthetic polymers; and,
at least one antigen.

2. The microparticle composition of claim 1 wherein the antigen is selected from the group consisting of foreign antigens and self-antigens.

3. The microparticle composition of claim 1 wherein the antigen is a protein antigen.

4. The microparticle of claim 3 wherein the protein antigen is an immunoglobulin or an immunoglobulin-like molecule.

5. The microparticle composition of claim 1 wherein the microparticle composition suppresses an ongoing deleterious immune response.

6. The microparticle composition of claim 1 wherein the microparticle composition prevents a deleterious immune response.

7. The microparticle composition of claim 1 wherein the microparticle composition prevents, suppresses or limits an immune response against a delivered bioactive payload.

8. The microparticle composition of claim 7 wherein the payload is a peptide hormone.

9. The microparticle composition of claim 1 wherein the microparticle composition enhances induction of a Th2 cellular response.

10. The microparticle composition of claim 9 wherein the microparticle composition induces an enhanced expression of IL-4.

11. The microparticle composition of claim 1 wherein the microparticle composition enhances induction of a humoral response.

12. The microparticle composition of claim 11 wherein the humoral response is directed against a foreign epitope or tumor associated antigens.

13. The microparticle of claim 12 wherein the foreign epitope is selected from the group consisting of microbial epitopes and parasitic epitopes.

14. The microparticle composition of claim 1 wherein the microparticle is compatible with deep lung delivery.

15. The microparticle of claim 1 wherein the surfactant is selected from the group consisting of phosphatides, non-ionic surfactants, cationic surfactants, proteins, amino acids and oligoaminoacids.

16. The microparticle of claim 8 wherein the phosphatide surfactant is chosen from the group consisting of homo and heterochain PC's, PS's, PE's, PG's, PI's, sphingomyelins, gangliosides, TAP's and DAP's, having one or two hydrocarbon chain length ranging from 5 to 22 carbon atoms.

17. The microparticle of claim 8 wherein the phosphatides may be hydrogenated, unsaturated or partially hydrogenated.

18. The microparticle of claim 17 wherein the phosphatides are phosphatides derived from soy or egg.

19. The microparticle composition of claim 15 wherein the phosphatide is selected from the group consisting of DiC18PC, DiC16PC, DiC14PC, DiC8PC DiC6PC, DiC16PS DiC14PS, DiC8PS and DiC6PS.

20. The microparticle composition of claim 15 wherein the non-ionic surfactant is selected from the group consisting of poloxamers, tweens, tritons, PEGs, and sugar esters.

21. The microparticle composition of claim 15 wherein the non-ionic surfactant is selected from the group consisting of poloxamer 188, poloxamer 407, tween 80, PEG 1540, cetyl alcohol and tyloxapol.

22. The microparticle composition of claim 15 wherein the non-ionic surfactant is selected from the group consisting of benzalkonium chloride, cholate, deoxycholates, CHAPs, taurocholate, deoxytaurocholate, phosphate fatty acid salts like dicetyl phosphate.

23. The microparticle composition of claim 1 wherein the at least one surfactant is selected from the group consisting of albumin, leucine, oligopeptides, oligoleucine, oligoalanine and saponins.

24. The microparticle composition of claim 1 wherein the carbohydrate excipient is selected from the group consisting of include hetastarch, starches, lactose, mannitol, mannose, inulin, mannan, sorbitol, galactitol, sucrose, trehalose, raffinose, maltose, glucose, cellulose and derivatives, pectins, dextrans, dextrins, chitosan, chitin, mucopolysaccharides, chondroitin sulfate and saponins.

25. The microparticle composition of claim 1 wherein the protein excipient is selected from the group consisting of human, egg or bovine albumin, chollagen, oligopeptides, oligoleucine, oligoalanine, gelatin, and glycoproteins.

26. The microparticle composition of claim 1 wherein the synthetic polymer excipient is selected from the group consisting of PLGA's, polylactides, polyglycolides, PVA's, PVP's, polyacrylics, carbomers, polyanhydrides, polyphosphoethers, polyurethanes, polyesters and polyphosphazenes.

27. The microparticle composition of claim 1 wherein the microparticle composition is delivered to the respiratory tract.

28. The microparticle composition of claim 1 wherein the antigen is insulin.

29. The microparticle composition of claim 1 wherein the formulated antigen, antigen fragment or antigen integrated into a recombinant molecule is disease associated and selected from the group consisting of insulin, GAD, HSP, collagen, MBP, PLP, and MOG.

30. The microparticle composition of claim 1 wherein the formulated antigen, antigen fragment or antigen integrated into a recombinant molecule is microbial associated selected from the group of microbes consisting of influenza, HIV, rotavirus, respiratory syncitial virus, hepatitis B, A, C, D, poliovirus, measles, mycobacteria tuberculosis, leishmania, listeria, pseudomonas, streptococcus and meningoccocus.

31. The microparticle composition of claim 1 further comprising tyloxapol.

32. A microparticle composition for the treatment of an autoimmune disorder comprising:
at least one surfactant wherein the at least one surfactant comprises approximately 1-80% of the total weight of the microparticle composition;
a carbohydrate that binds to the lectin receptors on antigen presenting cells; and
an antigen.

33. The microparticle composition of claim 32 wherein the autoimmune disorder is type 1 diabetes.

34. The microparticle composition of claim 32 wherein the main surfactant is a phosphatide.

35. The microparticle of claim 32 wherein the main surfactant is a phosphatidylcholine.

36. The microparticle composition of claim 35 wherein the main surfactant is a partially or hydrogenated phosphatidylcholine from egg or soy.

37. The microparticle composition of claim 32 wherein the lectin receptor is a mannose receptor.

38. The microparticle composition of claim 37 wherein the carbohydrate is mannan.

39. The microparticle composition of claim 32 wherein the antigen is insulin.

40. The microparticle composition of claim 39 wherein the antigen is insulin B chain.

41. The microparticle composition of claim 32 wherein the carbohydrate includes microbial or synthetic carbohydrates or derivatives.

42. A method of treating a patient suffering from Type 1 diabetes by administration of a therapeutically effect amount of microparticles as described in claim 32.

43. A method if treating a patient suffering from Type 1 diabetes by administration of a therapeutically effective amount of microparticles as described in claim 38.

44. The method of claim 42 wherein the patient is treated during the early initiation phase of insulitis.

45. The method of claim 42 wherein the patient is treated during later pathogenic stages of Type 1 diabetes associated with active islet cell destruction.

46. A method of enhancing the Th2 response of an individual suffering from an autoimmune disorder comprising administration of a therapeutically effective amount of the microparticle composition of claim 32.

47. A method of enhancing the Th2 response of an individual suffering from an autoimmune disorder comprising administration of a therapeutically effective amount of the microparticle composition of claim 40.

48. A method of enhancing the IL-4 production of an individual suffering from an autoimmune disorder comprising administration of a therapeutically effective amount of the microparticle composition of claim 32.

49. A method of tolerizing pathogenic T-cells in an individual suffering from autoimmune diabetes comprising administration of a therapeutically effective amount of the microparticle composition of claim 37.

50. A method of preventing the onset of Type 1 diabetes by administration of a therapeutically effective amount of the microparticle composition of claim 32.

51. A microparticle composition for the treatment of an autoimmune disorder comprising:
a surfactant or surfactant mixture comprising approximately 1-80% of the total weight of the microparticle composition; and
a carbohydrate that binds to the lectin receptors on antigen presenting cells comprising approximately 1-60% of the total weight of the microparticle composition.

52. The microparticle composition of claim 51 wherein the main surfactant is a phosphatide.

53. The microparticle composition of claim 51 wherein the lectin receptor is a mannose receptor.

54. The microparticle composition of claim 51 wherein the carbohydrate is mannan.

55. The microparticle composition of claim 51 wherein the main surfactant is a partially or hydrogenated phosphatidylcholine from egg or soy.

56. A method of preventing the development of type 1 diabetes comprising administering a therapeutically effective amount of the microparticle compositions of claim 51.

57. A method of preventing the development of type 1 diabetes comprising administering a therapeutically effective amount of the microparticle composition of claim 53.

58. A method of preventing the development of type 1 diabetes comprising administering a therapeutically effective amount of the microparticle composition of claim 54.

59. A method of enhancing the Th2 response of an individual suffering from an autoimmune disorder comprising administration of a therapeutically effective amount of the microparticle composition of claim 51.

60. A method of tolerizing pathogenic T-cells in an individual suffering from autoimmune diabetes comprising administration of a therapeutically effective amount of the microparticle composition of claim 51.

61. A microparticle composition for delivering a bioactive substance where it is desired to limit the immune response to the bioactive substance comprising:
a water soluble surfactant selected from the group consisting of: phosphatides, non-ionic surfactants, anionic surfactants, cationic surfactants, proteins, amino acids and oligoaminoacids;
a water soluble excipient comprising a weight ratio of 1-90% of the total weight of the composition wherein the water soluble excipients is selected from the group consisting of lactose, mannitol, mannose, sorbitol, galactitol, sucrose, trehalose, raffinose, maltose, glucose, saponins, osmotic agents such as sodium chloride, potassium chloride, calcium chloride, magnesium chloride, zinc chloride, buffers such as PBS, acetate, citrate, TRIS and amino acids such as glycine and alanine; and,
a bioactive substance.

62. The microparticle composition of claim 61 wherein the bioactive substance is insulin.

63. The microparticle composition of claim 61 wherein the microparticle composition is administered to the respiratory tract.

64. The microparticle composition of claim 61 wherein the microparticle composition results in a less retentive microparticle which has a high dissolution rate in water.

65. The microparticle composition of claim 61 wherein the microparticle composition results in a high dissolution rate and a low clearance by phagocytes in the respiratory tract.

66. The microparticle composition of claim 61 wherein the microparticle composition limits the Th2 immune response.

67. The microparticle of claim 61 further comprising a metal ion.

68. The microparticle composition of claim 61 further comprising tyloxapol.

69. The microparticle of claim 61 wherein the surfactant or mixture of surfactants is present in an amount of approximately 1-80% of the total weight of the microparticle composition.

70. A microparticle composition for delivering a bioactive substance where it is desired to enhance the immune response to the bioactive substance comprising:
a surfactant or mixture of surfactants selected from the group consisting of phosphatides, non-ionic surfactants, anionic surfactants, cationic surfactants, proteins, amino acids and oligaminoacids;
an excipient selected from the group consisting of starches, lactose, mannitol, mannose, inulin, mannan, sorbitol, galactitol, sucrose, trehalose, raffinose, maltose, glucose, cellulose and derivatives, pectins, dextrans, dextrins, chitosan, chitin, mucopolysaccharides, chondroitin sulfate, saponins osmotic agents such as sodium chloride, potassium chloride, calcium chloride, magnesium chloride, zinc chloride, buffers such as PBS, acetate, citrate, TRIS, amino acids such as glycine and alanine, human, egg or bovine albumin, chollagen, oligopeptides, oligoleucine, oligoalanine, gelatin, glycoproteins, PLGA's, polylactides, polyglycolides, PVA's, PVP's, polyacrylics, carbomers, polyanhydrides, polyphosphoethers, polyurethanes, polyesters and polyphosphazenes;
and a bioactive substance for inducing an immune response.

71. The microparticle composition of claim 70 wherein the microparticle composition is administered to the respiratory tract.

72. The microparticle composition of claim 70 wherein the bioactive substance is insulin.

73. The microparticle composition of claim 70 wherein the microparticle composition results in a retentive microparticle which slows the release of the bioactive substance.

74. The microparticle composition of claim 70 wherein the microparticle composition results in aggregation and slows clearance by phagocytes in the respiratory tract.

75. The microparticle composition of claim 70 wherein the microparticle composition increases the Th2 immune response.

76. The microparticle of claim 70 further comprising a metal ion.

77. The microparticle composition of claim 70 wherein a non-ionic surfactant is added to increase the release rate of the bioactive substance.

78. The microparticle composition of claim 77 wherein the non-ionic surfactant is tyloxapol.

79. The microparticle composition of claim 70 wherein the bioactive substance is selected from the group consisting of nucleic acids, nucleotides, peptides and proteins.

80. The microparticle composition of claim 1 wherein the microparticle composition can be administered to the respiratory tract by liquid dose instillation, nebulization, aerosolization, dry powder inhalation and metered dose instillation.
